(19) United States
(12) Patent Application Publication
(10) Pub. No.: US 2026/0014077 A1
(43) Pub. Date: Jan. 15, 2026

(54) NOVEL METHODS TO ENHANCE GENE DELIVERY

(71) Applicant: TIBA Biotech LLC, Cambridge, MA (US)

(72) Inventors: Heliang Song, Winchester, MA (US); Poulami Talukder, Medford, MA (US)

(21) Appl. No.: 19/265,612

(22) Filed: Jul. 10, 2025

Related U.S. Application Data
(60) Provisional application No. 63/669,486, filed on Jul. 10, 2024.
(60) Provisional application No. 63/669,505, filed on Jul. 10, 2024.

(51) Int. Cl. A61K 9/1271; A61K 9/1277; A61K 9/51; A61K 39/00; A61K 39/385

(52) U.S. Cl. CPC A61K 9/1271; A61K 9/1277; A61K 9/5123; A61K 9/5192; A61K 39/385; A61K 2039/53; A61K 2039/55555; A61K 2039/645

(57) ABSTRACT

Disclosed are nanoparticle preparations including a nucleic acid agent, a nucleic acid carrier, a metal salt, a polyethylene glycol (PEG)-lipid, a phospholipid, and a cholesterol or its derivative. Also provided are preparation methods and methods for treating or preventing a condition in a subject using such a nanoparticle preparation.

### CROSS REFERENCE TO RELATED APPLICATIONS

This application claims the benefit of priority to U.S. Application Ser. Nos. 63/669,486 and 63/669,505, both filed on Jul. 10, 2024. The entire contents of both applications are incorporated by reference.

### FIELD OF INVENTION

The disclosure relates to nanoparticle preparations for nonviral gene delivery and methods of treating disorders.

### BACKGROUND

During the 2019 pandemic, RNA vaccines demonstrated great success in protecting the general population from serious infections. A stable and effective delivery system is required to protect RNAs in vivo from nuclease degradation before they enter target cells in living tissue. Lipid nanoparticles (LNPs), as an effective non-viral delivery tool, have been used to encapsulate RNAs to protect them from nuclease degradation and achieve sufficient cellular transfection to generate an immune response for vaccine applications.

LNPs have indeed shown the potential in disease treatment due to recent progress in nucleic acid therapy. However, there are barriers limiting the therapeutic benefits of these delivery systems, including (i) low endosomal escape rate and (ii) extra hepatic delivery. As reported in Zheng et al., Proceedings of the National Academy of Sciences, 120 (27), e2301067120 (2023), only 2% of the siRNA encapsulated in LNP is released from the endosome to the cytosol.

There is a need to design nanoparticle formulations to enhance endosomal escape and thus improve the therapeutic effect of RNA medicines. An ideal nanoparticle preparation should be easy to apply to a wide range of applications with low cost and minimal side effects.

Another barrier that these delivery tools need to overcome for effective disease treatment is to develop non-hepatic formulations since most of the current carrier systems (e.g. LNPs) are liver tropic, limiting their application to liver-specific diseases. Based on recently published work, brain and heart remain very difficult to target via IV administration. Alternatively, injecting them directly into organs, requires more invasive procedure which is not ideal. Therefore, modifications of the composition of nanoparticles to target vital organs with high specificity are needed for clinical use.

### SUMMARY

This invention is based on the unexpected discovery that metal salts can greatly increase the delivery efficiency of nanoparticles by increasing osmotic pressure, fusing with endosomal membrane lipids, and thus assisting endosomal escape.

This invention also shows that a metal salt incorporation into nanoparticle preparation may lead to altered organ tropism, and thus different biodistribution profile.

In one aspect, the invention relates to a nanoparticle preparation containing a nucleic acid agent, a nucleic acid carrier, a metal salt, a polyethylene glycol (PEG)-lipid, a phospholipid, and a cholesterol or derivative thereof.

The nanoparticle preparation of this invention has one or any combination of the following features:
1. the metal salt has a divalent or trivalent metal ion;
2. the nucleic acid agent is a polynucleotide, oligonucleotide, a deoxyribonucleic acid (DNA), a complementary DNA (cDNA), a ribonucleic acid (RNA), a replicon RNA (repRNA), a small interfering RNA (siRNA), a microRNA (miRNA), a single strand guide RNA (sgRNA), a messenger RNA (mRNA), or any combination thereof;
3. the nucleic acid agent can encode a protein, a polypeptide, or an oligopeptide, the protein being a therapeutic protein or an immunogenic protein in which the immunogenic protein comprises one or more antigens associated with an infectious disease, a pathogen, a cancer, an autoimmune disease, or an allergic disease, and the therapeutic protein is effective against an infectious disease, a pathogen, a cancer, an autoimmune disease, or an allergic disease. Infectious diseases include those associated with severe acute respiratory syndrome coronavirus 2 (SARS-COV-2), the causative agent of coronavirus disease 2019 (COVID-19), respiratory syncytial virus (RSV), Zika virus, influenza virus, Mycobacterium tuberculosis, and parasites of the genus Plasmodium. Examples of cancers from which the antigen may be derived include melanoma, non-small cell lung cancer (NSCLC), and triple-negative breast cancer (TNBC). Autoimmune diseases or disorders include type 1 diabetes, rheumatoid arthritis and psoriasis. Allergic disease includes food allergies, asthma and atopic dermatitis;
4. the nucleic acid agent can be an RNA or DNA capable of silencing, inhibiting, or modifying the activity of a gene, for example, a gene-editing complex;
5. the nucleic acid carrier is a dendrimer or dendron;
6. the dendrimer or dendron has an amine group;
7. the PEG-lipid is 1,2-dimyristoyl-sn-glycero-3-phosphoethanolamine-N-[methoxy (polyethylene glycol)-2000], 1,2-dimyristoyl-rac-glycero-3-methoxypolyethylene glycol-2000, or any combination thereof;
8. the PEG-lipid constitutes 1% to 10% (preferably 1% to 5%) by weight of the nanoparticle preparation;
9. the phospholipid is 1,2-dioleoyl-sn-glycero-3-phosphoethanolamine (DOPE), distearoylphosphatidylcholine (DSPC), or a combination thereof;
10. the phospholipid constitutes 1% to 25% (preferably 1% to 15%) by weight of the nanoparticle preparation;
11. the cholesterol or its derivative constitutes 1% to 50% (preferably 5% to 25%) by weight of the nanoparticle preparation;
12. the metal salt is selected from the group consisting of calcium chloride, magnesium chloride, manganese chloride, barium chloride, copper chloride, zinc chloride, calcium hydroxide, magnesium hydroxide, manganese hydroxide, barium hydroxide, copper hydroxide, zinc hydroxide, calcium sulfate, magnesium sulfate, manganese sulfate, barium sulfate, copper sulfate, zinc sulfate, aluminum chloride, aluminum sulfate, aluminum hydroxide, ferric chloride, ferric sulfate, ferric hydroxide, and any combinations thereof;
13. the metal salt constitutes 0.01% to 90% (preferably, 0.1% to 55%) by weight of the nanoparticle preparation; and
14. the metal salt is fully or partially encapsulated in the nanoparticle.

Another aspect of this invention relates to a pharmaceutical composition containing any one of the nanoparticle preparations described above and a pharmaceutically acceptable carrier.

Also within the scope of the invention is a method for treating or preventing a condition in a subject comprising administering to a subject in need thereof a therapeutically effective amount of any one of the nanoparticle preparations described above or their pharmaceutical compositions. The nucleic acid agent is administered to the subject in a range preferably from 0.001 ng nucleic acid to 10 mg nucleic acid per kg body weight of the subject. The subject can be a mammal selected from the group consisting of a human, a non-human primate, a mouse, a rat, a rabbit, a dog, a cat, a horse, a pig, a sheep, and a cow.

Still within the scope of the invention is a method of preparing the nanoparticle preparation. The method includes the following steps of: (a) providing an aqueous phase containing the nucleic acid agent, (b) providing a lipid phase containing the nucleic acid carrier, the PEG-lipid, the phospholipid, and the cholesterol or its derivative, and (c) mixing the aqueous phase and the lipid phase to form the nanoparticle, provided that the aqueous or lipid phase contains a metal salt. Each of the components is as described above.

The details of the invention are set forth in the drawings, the definitions, and the detailed description below. Other features, objects, and advantages of the invention will be apparent from the following actual examples and claims.

### BRIEF DESCRIPTION OF THE DRAWINGS

For the purpose of illustrating the invention, particular embodiments are shown in the drawings. In the drawings:

FIGS. 1a-1d illustrate the size distribution of the nanoparticle preparations measured as the intensity based on diameter (d) of the nanoparticle preparations.

FIG. 2 illustrates quantification in vitro of secreted alkaline phosphatase (SEAP) expression after administration of nanoparticle formulations based on Dendrimer-1 to living cells.

FIGS. 3a-3c illustrate quantification in vivo of SEAP expression after administration of nanoparticle preparations based on Dendrimer-2, Dendrimer-1, and Dendrimer-3, respectively, to mice.

FIGS. 4a-4c illustrate quantification in vivo of mouse serum IgG specific to PR8 HA protein after vaccination with PR8 HA replicon RNA formulated with nanoparticle preparations using Dendrimer-2, Dendrimer-1, and Dendrimer-3, respectively.

FIGS. 5a-5b illustrate quantification in vivo of Luciferase expression at different organs after administration of nanoparticle preparations containing Luc replicon RNA and Dendrimer-4 without and with CaCl₂), respectively.

FIG. 6 shows the morphology and internal structure of a nanoparticle preparation of this invention by Cryo-electron microscopy (CryoEM).

FIG. 7 indicates that a nanoparticle preparation containing calcium ions exhibited significantly greater RNA expression in the heart as compared to the lung, spleen, and liver.

### DETAILED DESCRIPTION

The nanoparticle preparations of this invention can be prepared following conventional methods such as any turbulent mixing methods including, but not limited to, microfluidic mixing, vortex mixing, and pipette mixing. See Wang et al., Nature Protocols 18, 265-291 (2023).

In a specific method, the nanoparticle preparation is prepared by mixing an aqueous phase and a lipid phase, e.g., in a microfluidic mixer. The aqueous phase contains a nucleic acid agent. The lipid phase contains a nucleic acid carrier, a PEG-lipid, a phospholipid, and cholesterol or a cholesterol derivative. Further, a metal salt is included in the aqueous phase, the lipid phase, or both.

The nanoparticle preparation thus prepared contains a plurality of nanoparticles, each of which is generally spherical in shape having a nominal diameter of 1 nm to 1000 nm, preferably 10 nm to 500 nm, and more preferably 25 nm to 250 nm. Exemplary sizes of the nanoparticles are 5 nm, 10 nm, 50 nm, 60 nm, 80 nm, 100 nm, 120 nm, 150 nm, 180 nm, 200 nm, 250 nm, 300 nm, and 500 nm.

A nanoparticle preparation is defined as a liquid suspension of nanoparticles in water or an aqueous buffer, or a dry mass of such nanoparticles generated by a drying process such as spray drying or lyophilization.

These nanoparticles each can have a size as described above. Their size and size distribution can be measured using known methods, e.g., dynamic light scattering (DLS) and electron microscopy.

A typical polydispersity index (PDI) of the nanoparticle preparation has a range of 0.0001 to 0.5 (e.g., 0.001 to 0.25, 0.002 to 0.2, 0.003 to 0.1, and 0.2 or less) as measured by the dynamic light scattering method.

Not to be bound by any theory, the nanoparticle can have a lipid core (e.g., a solid or dense lipid core), an aqueous core, or both, each of which is covered by an outer layer that stabilizes the core. The nucleic acid agent is encapsulated in the lipid or aqueous core, including partial encapsulation and full encapsulation. In the case of partial encapsulation, the lipid core covers the molecule(s) of the nucleic acid agent by at least 20% (e.g., at least 30%, at least 40%, at least 50%, at least 60%, at least 80%, and 50% to 100%). In the case of full encapsulation, the nucleic acid agent is localized completely inside the lipid core. In the context of nucleic acid immunogenic, vaccine or therapeutic agents, full encapsulation may be determined by a Ribogreen® assay developed by Molecular Probes, Inc. (Chelmsford, Massachusetts). RiboGreen® is an ultra-sensitive fluorescent nucleic acid stain for quantitating oligonucleotides and single-stranded DNA or RNA in a solution (available from Thermo Fisher Scientific, Waltham, Massachusetts).

It is possible that the nanoparticle has a bleb structure containing a bleb fused with a lipid droplet, in which the bleb has a bleb core enclosed by a lipid bilayer fused with the lipid droplet. See Cheng et al., Advanced Materials 2023, 35, 2303370. In the bleb structure, the nucleic acid agent is included in the bleb core. As explained in Cheng et al., the bleb structure can be prepared by (i) providing vesicles having a liquid core enclosed by a lipid bilayer, preferably with the vesicles being positively charged, (ii) providing lipid nanoparticles containing an ionizable lipid and a nucleic acid agent encapsulated in the lipid nanoparticles, (iii) fusing the vesicles and the lipid nanoparticles driven by a buffer of pH 4 or lower (e.g., 100 mM to 800 mM sodium citrate aqueous solution), thereby forming fused lipid nanoparticles containing the nucleic acid agent in the lipid core and the blebs each having a lipid bilayer and an empty liquid core, and (iv) neutralizing the ionizable lipid to induce phase separation of the lipid nanoparticles into lipid droplets and migrating the nucleic acid agent to the liquid core (i.e., the bleb core), thus obtaining the bleb structure containing the nucleic acid agent in the bleb core.

### Nucleic Acid Agents

The nucleic acid agent of the nanoparticle can be therapeutic or immunogenic. The terms “nucleic acid” and “nucleic acid agent” are used herein interchangeably.

Exemplary nucleic acid agents include RNAs, DNA, and any combinations thereof. The term “DNA” or “DNA molecule” or “deoxyribonucleic acid molecule” refers to a polymer of deoxyribonucleotides. The DNA molecule may be a polynucleotide, oligonucleotide, DNA, or cDNA. The DNA molecule may encode wild-type or engineered proteins, peptides or polypeptides. The encoded protein, peptide, or polypeptide may be an antigen. The term “RNA” or “RNA molecule” or “ribonucleic acid molecule” refers to a polymer of ribonucleotides. The polymer may have 2, 3, 4, 5, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000 or more ribonucleotides. The polymer may have 2, 3, 4, 5, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, or 50000 ribonucleotides, or a number of ribonucleotides in a range between any two of the foregoing. The RNA molecule may be a replicon RNA (repRNA), small interfering RNA (siRNA), miRNA, single strand guide RNA (sgRNA), messenger RNA (mRNA), or transfer RNA (tRNA). The term “replicon RNA” (repRNA) refers to a virus-derived nucleic acid genome that may be replication-competent, or progeny-defective (i.e. incapable of producing infectious progeny virions), that undergoes self-copying when introduced into a cell. Viral genomes that are typically modified for use as repRNAs include “positive strand” RNA viruses. The modified viral genomes function as both mRNA and templates for replication. The term “small interfering RNA” (siRNA) refers to an RNA (or RNA analog) comprising between about 10-50 nucleotides (or nucleotide analogs) which is capable of directing or mediating RNA interference. The term “microRNA” (miRNA) refers to a small (20-24 nt) regulatory non-coding RNA that is involved in post-transcriptional regulation of gene expression in eukaryotes by affecting either or both the stability and translation of coding mRNAs. The term “messenger RNA” (mRNA) is a single-stranded RNA and defines the amino acid sequence of one or more polypeptide chains. This information is translated during protein synthesis when ribosomes bind to the mRNA. The DNA or RNA molecules may be chemically modified in the nucleic acid backbone, the ribose sugar moiety, and/or the nucleobase itself.

The therapeutic or immunogenic nucleic acid agent may be non-covalently bound or covalently bound to the nucleic acid carrier. The therapeutic or immunogenic agent may be a nucleic acid agent bound to the charged nucleic acid carrier through electrostatic interaction. The nucleic acid agent may be bound to the charged nucleic acid carrier through electrostatic interaction or hydrogen bonding.

The immunogenic or therapeutic nucleic acid agents can encode antigens. “Antigen” as used herein is defined as a molecule that triggers an immune response. The immune response may involve either antibody production, or the activation of specific immunologically active cells, or both. The antigen may refer to any molecule capable of stimulating an immune response, including macromolecules. In an embodiment, the macromolecules are proteins, peptides, or polypeptides. The antigen may be a structural component of a pathogen, or a cancer cell or a derivative thereof. The antigen may be synthesized, produced recombinantly in a host, or may be derived from a biological sample, including but not limited to a tissue sample, cell, or a biological fluid.

Examples of an antigen include but are not limited to a vaccine antigen, parasite antigen, bacterial antigen, tumor antigen, environmental antigen, therapeutic antigen or an allergen. As used herein a nucleotide vaccine is a DNA- or RNA-based prophylactic or therapeutic composition capable of stimulating an adaptive immune response in the body of a subject by delivering antigen(s). The immune response induced by vaccination typically results in development of immunological memory, and the ability of the organism to quickly respond to subsequent encounter with the antigen or infectious agent.

The nanoparticle preparations contain the nucleic acid agent in a pharmaceutically effective concentration that is required to confer a therapeutic effect or other benefits. Effective concentrations depend on various conditions, e.g., species of the nucleic acid used, concentration of the nucleic acid carrier, route of administration, condition as treated, etc. A skilled person can determine the actual concentration following conventional protocols used in the art.

The nanoparticle preparations may be used for delivery of therapeutic or immunogenic nucleic acids for gene targeting. The therapeutic or immunogenic nucleic acid may be an antisense oligonucleotide (AON) or a double-stranded small interfering RNA (siRNA). Typically, siRNAs are between 21 and 23 nucleotides in length. The siRNAs may comprise a sequence complementary to a sequence contained in an mRNA transcript of a target gene when expressed within the host cell. The antisense oligonucleotide may be a morpholino antisense oligonucleotide. The antisense oligonucleotide may include a sequence complementary to a sequence contained in an mRNA transcript of a target gene. The therapeutic or immunogenic nucleic acid may be an interfering RNA (iRNA) against a specific target gene within a specific target organism.

The siRNA may induce sequence-specific silencing of the expression or translation of the target gene, thereby down-regulating or preventing gene expression. The siRNA may completely inhibit expression of the target gene. The siRNA may reduce the level of expression of the target gene compared to that of an untreated control. The therapeutic or immunogenic nucleic acid may be a microRNA (miRNA). The miRNA may be a short RNA, e.g., a hairpin RNA (hpRNA). The miRNA may be cleaved into biologically active dsRNA within the target cell by the activity of the endogenous cellular enzymes. The RNA may be a double stranded RNA (dsRNA). The ds RNA may be at least 25 nucleotides in length or may be longer. The dsRNA may contain a sequence that is complementary to the sequence of the target gene or genes. An embodiment comprises use of a nanoparticle preparation for gene targeting in a subject. An embodiment comprises a method of gene targeting comprising administering a nanoparticle preparation herein to a subject.

In an embodiment, the therapeutic or immunogenic nucleic acid agent may be or may encode an agent that totally or partially reduces, inhibits, interferes with, or modulates the activity or synthesis of one or more genes encoding the protein or proteins of interest. The target genes may be any gene in the genome of a host organism. The sequence of the therapeutic or immunogenic nucleic acid may not be 100% complementary to the nucleic acid sequence of the target gene.

In an embodiment, the nanoparticle preparation may be used for targeted, specific alteration of the genetic information in a subject. An embodiment comprises targeted, specific alteration of the genetic information in a subject comprising administration of a nanoparticle preparation herein. As used herein, the term “alteration” refers to any change in the genome in the cells of a subject. The alteration may be insertion or deletion of nucleotides in the sequence of a target gene. “Insertion” refers to addition of one or more nucleotides to a sequence of a target gene. The term “deletion” refers to a loss or removal of one or more nucleotides in the sequence of a target gene. The alteration may be correction of the sequence of a target gene. “Correction” refers to alteration of one or more nucleotides in the sequence of a target gene, e.g., by insertion, deletion, or substitution, which may result in a more favorable expression of the gene manifested by improvements in genotype and/or phenotype of the host organism. An embodiment comprises use of a nanoparticle preparation herein for targeted, specific alteration of the genetic information in a subject. An embodiment comprises a method of targeted, specific alteration of the genetic information in a subject comprising administering a nanoparticle preparation herein to the subject. An embodiment comprises use of a nanoparticle preparation herein for the alteration of the genetic information in the cells of a subject ex vivo by administration of the nanoparticle preparation directly to the solution in which the subject's cells are cultured or suspended. The alteration of the genetic information may be achieved via genome editing techniques. As used herein, “genome editing” refers to the process of modifying the nucleotide sequence in the genome in a precise or controlled manner.

An exemplary genome editing system is a Clustered Regularly Interspaced Short Palindromic Repeats (CRISPR) system as described, for example, in WO 2018/154387, which published Aug. 30, 2018 and is incorporated herein by reference as if fully set forth. In general, “CRISPR system” refers to transcripts and other elements involved in the expression of CRISPR-associated (Cas) genes, including sequences encoding a Cas gene, a tracr (trans-activating CRISPR) sequence, a tracr-mate sequence, a guide sequence, or other sequences and transcripts from a CRISPR locus. One or more tracr mate sequences may be operably linked to a guide sequence before processing or crRNA after processing by a nuclease. The tracrRNA and crRNA may be linked and may form a chimeric crRNA-tracrRNA hybrid where a mature crRNA is fused to a partial tracrRNA via a synthetic stem loop to mimic the natural crRNA:tracrRNA duplex as described in Cong et al., Science, 15:339 (6121): 819-823 (2013) and Jinek et al., Science, 337 (6096): 816-21 (2012), which are incorporated herein by reference as if fully set forth. A single fused crRNA-tracrRNA construct is also referred herein as a guide RNA or gRNA, or single-guide RNA (sgRNA). Within an sgRNA, the crRNA portion is identified as the “target sequence” and the tracrRNA is often referred to as the “scaffold.” In an embodiment, the nanoparticle preparations described herein may be used to deliver an sgRNA.

In an embodiment, the nanoparticle preparations may be used to apply other exemplary genome editing systems including meganucleases, homing endonucleases, TALEN-based systems, or Zinc Finger Nucleases. The nanoparticle preparations may be used to deliver the nucleic acid (RNA and/or DNA) that encodes the sequences for these gene editing tools, and the actual gene products, proteins, or other molecules.

An embodiment comprises use of a nanoparticle preparation herein for genome editing in a subject. An embodiment comprises a method of genome editing in a subject comprising administering a nanoparticle preparation herein to the subject. The nucleic acid in these embodiments may be a sgRNA. The nucleic acid in these embodiments may be one for genome editing via meganucleases, homing endonucleases, TALEN-based systems, or Zinc Finger Nucleases.

In an embodiment, the nanoparticle preparation may be used for gene targeting in a subject in vivo or ex vivo, e.g., by isolating cells from the subject, editing genes, and implanting the edited cells back into the subject. An embodiment comprises a method comprising administering a nanoparticle preparation herein to isolated cells from a subject. The method may include gene targeting. The method may comprise implanting the edited cells back into the subject (or into another subject).

### Nucleic Acid Carriers

The nanoparticle of this invention further contains one or more nucleic acid carriers. The nucleic carriers can be dendrimers or dendrons. As used herein, a “dendrimer” is a molecular architecture with an interior core (i.e., a dendritic skeleton) and layers (or “generations”) of repeating units which are attached to and extend from this interior core, each layer having one or more branching points, and an exterior surface of terminal groups attached to the outermost generation. Dendrimers have regular dendritic or “starburst” molecular structures. A dendritic skeleton may comprise 2,2-bis(hydroxymethyl) propionic acid or 2,2-Bis(hydroxymethyl) butyric acid as the monomeric polyester unit. A “dendron” refers to a dendrimer containing an interior core that has a focal point chemically addressable by one or more layers (or “generations”) of repeating units.

Suitable dendrimers include those described in Talukder et al., International Application Publication No. WO2021207020A1 and WO 2022/212838 A1, which is incorporated herein by reference as if fully set forth.

A preferred nucleic acid carrier has a dendritic skeleton, an amine group or an amine linker, and a hydrophobic unit.

More preferably, the nucleic acid carrier has a structure of formula Ia or Ib:

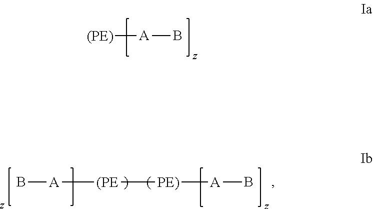

wherein PE is a polyester dendrimer or dendron which includes a core and a plurality of monomeric polyester units that form one or more generations, A is an amine linker, B is a hydrophobic unit, and z is the number of surface groups.

PE can have the structure of Formula II:

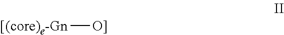

wherein c is the core multiplicity or number of wedges originating from the core, whose values independently range from 1 to 6, G is a layer or generation of dendrimer or dendron and n is a generation number in a range from 1 to 10.

Referring to formula Ia and formula Ib above, the nucleic acid carrier has one or more of the following features:
(i) the monomeric polyester unit of the plurality is 2,2-bis(hydroxymethyl) propionic acid or 2,2-bis(hydroxymethyl) butyric acid,
(ii) z has Formula III: cbⁿ (III), wherein b is branch point multiplicity, or number of branches at each branching point; c is the core multiplicity or number of wedges originating from the core in a range from 1 to 6, and n is a generation number in a range from 1 to 10,
(iii) c is 1, and the core is a unidirectional core, wherein the unidirectional core can be a carboxylic acid or derivative thereof, preferably selected from the group consisting of:

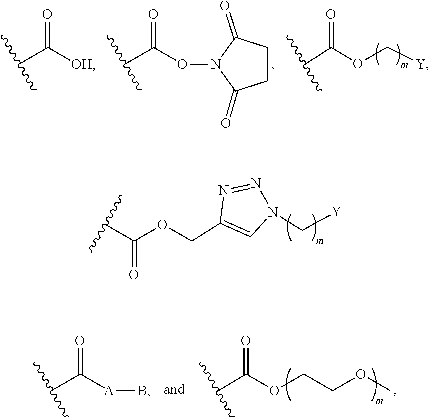

in which Y is selected from methyl, iso-propyl, sec-butyl, iso-butyl, tert-butyl, isopentyl, neopentyl, 3-methylhexyl, 2,2-dimethylpentyl, 2,3-dimethylpentyl, azide (N3), halogen (Cl, Br, or I), acetylene (C₂H₂), hydroxyl (—OH), or thiol (—SH), -pyranosyl, cycloalkyl, aryl, heteroaryl, and heterocycle; A is an amine linker; B is a hydrophobic unit; and m is 1 to 20, the cycloalkyl, aryl, heteroaryl, and heterocycle are unsubstituted or substituted with at least one group selected from halogen, hydroxyl (—OH) and alkyl group,
(iv) c is 3, and the core is a three directional core, which is preferably trimethylol propane, or 1,1,1-tris(hydroxyphenylethane), and has the structure of:

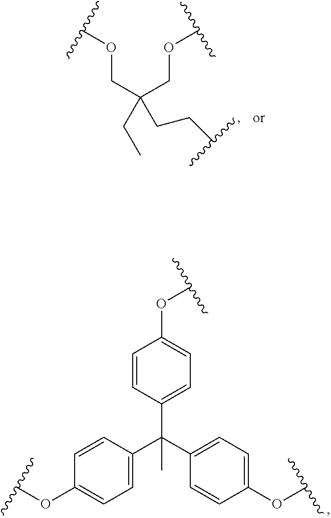 [SMILES: *CCC(CC)(CO*)CO* ; *Oc1ccc(C(C)(c2ccc(O*)cc2)c2ccc(O*)cc2)cc1]

respectively,

(v) c is 4, and the core is a four directional core preferably selected from the group consisting of: pentaerythritol, adamantane-1,3,5,7-tetraol, 5,10,15,20-tetrakis(4-hydroxy-phenyl)-21H,23H-porphine, [1,1′-biphenyl]-3,3′,5,5′-tetraol, 2,3,6,7-tetrahydroxy-9,10-di-methylanthracene, 3,9,10-dimethyl-9,10-dihydro-9,10-ethanoanthracene-2,3,6,7-tetraol, 4,6,13-dihydropentacene-5,7,12,14-tetraol, hexahydro-[1,4]dioxino[2,3-b][1,4]dioxine-2,3,6,7-tetraol, anthracene-1,4,9,10-tetraol, pyrene-1,3,6,8-tetraol, and 3,3,3′,3′-tetramethyl-2,2′,3,3′-tetrahydro-1,1′-spirobi[indene]-5,5′,6,6′-tetrol, and has the structure of:

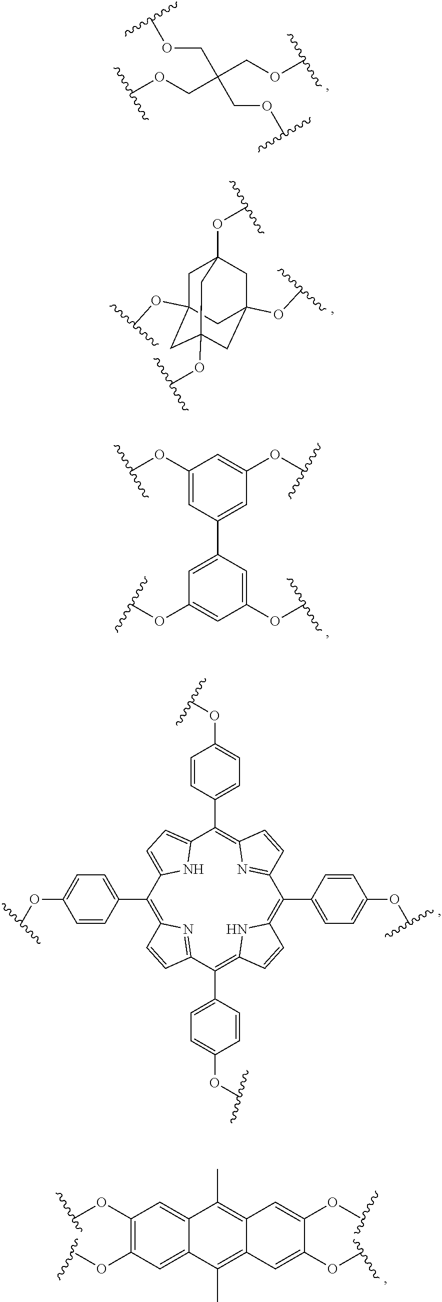

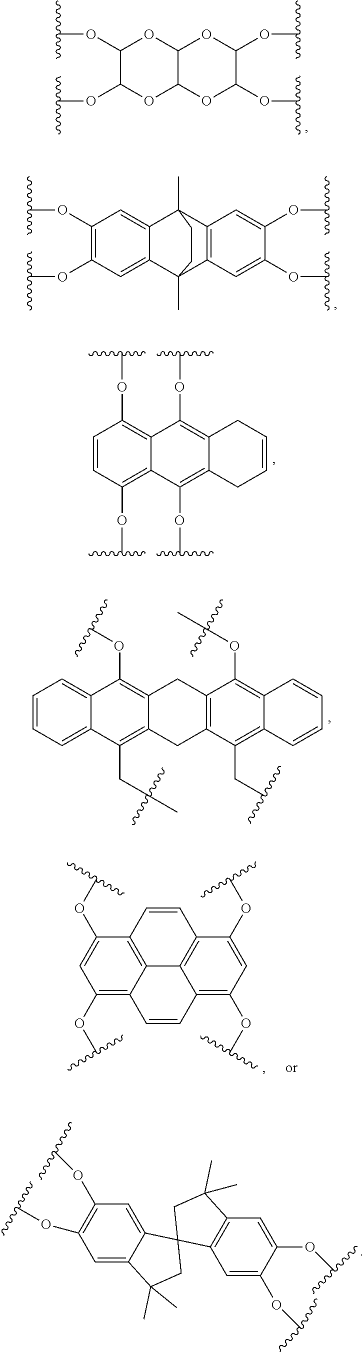

respectively,

(vi) A is derived from the group consisting of: N1-(2-aminoethyl)ethane-1,2-diamine, N1-(2-aminoethyl)propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-amino-ethyl)ethane-1,2-diamine), N1-(2-(4-(2-aminoethyl)piperazin-1-yl)ethyl)ethane-1,2-diamine, N1-(2-aminoethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl) (methyl)amino)propan-1-ol, 3,3′-(methylazanediyl)bis(propan-1-ol), N1-(3-aminopropyl)-N1-methylbutane-1,4-diamine, 4-((3-aminopropyl)(methyl)amino)butan-1-ol, 4-((3-hydroxy-propyl)(methyl)amino)butan-1-ol, 4-((3-hydroxypropyl)(methyl)amino)butan-1-ol, N1-(4-aminobutyl)-N1-methylbutane-1,4-diamine, 4-((4-aminobutyl)(methyl)amino)butan-1-ol, 4,4′-(methylazanediyl)bis(butan-1-ol), 3-((3-aminopropyl)(ethyl)amino)propan-1-ol, 3,3′-(ethylazanediyl)bis(propan-1-ol), N1-(3-aminopropyl)-N1-ethylbutane-1,4-diamine, 4-((3-aminopropyl)(ethyl)amino)butan-1-ol, 4-ethyl(3-hydroxypropyl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl)bis(butan-1-ol), 3-((3-aminopropyl)amino)propan-1-ol, N1-(3-aminopropyl)butane-1,4-diamine, 4-((3-hydroxypropyl)amino)butan-1-ol, N1-(4-amino-butyl)butane-1,4-diamine, 3,3′-azanediylbis(propan-1-ol), 4-((3-aminopropyl)amino)butan-1-ol, 4,4′-azanediylbis(butan-1-ol), and N1,N1′-(butane-1,4-diyl)bis(propane-1,3-diamine); and has the structure of:

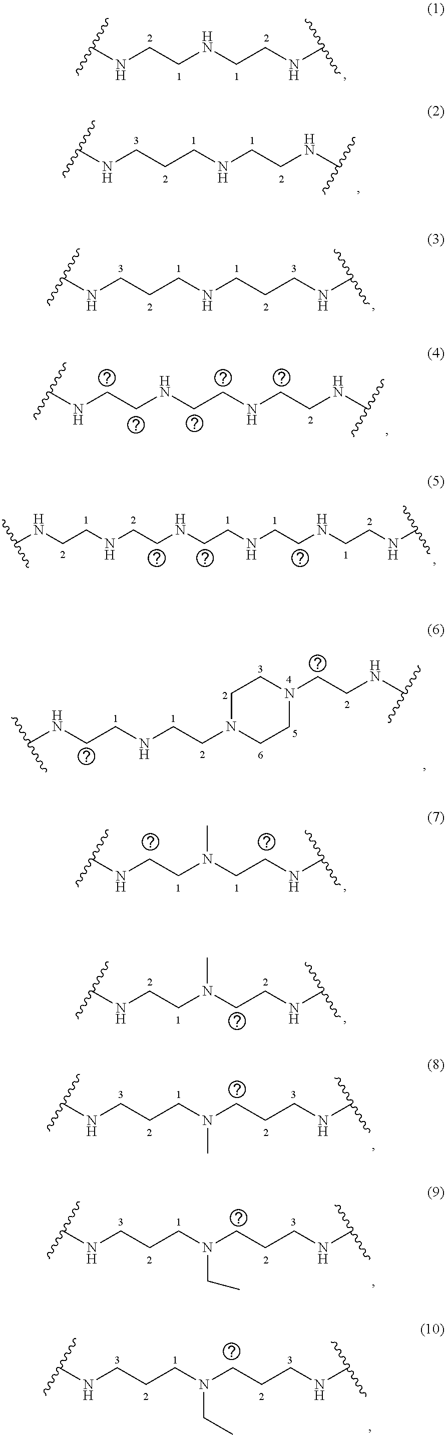

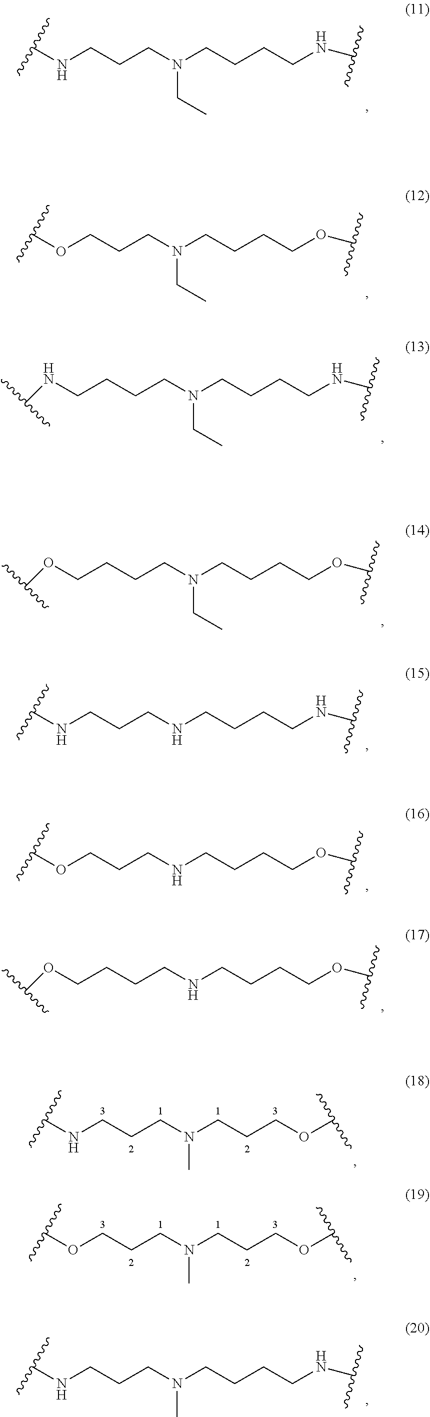

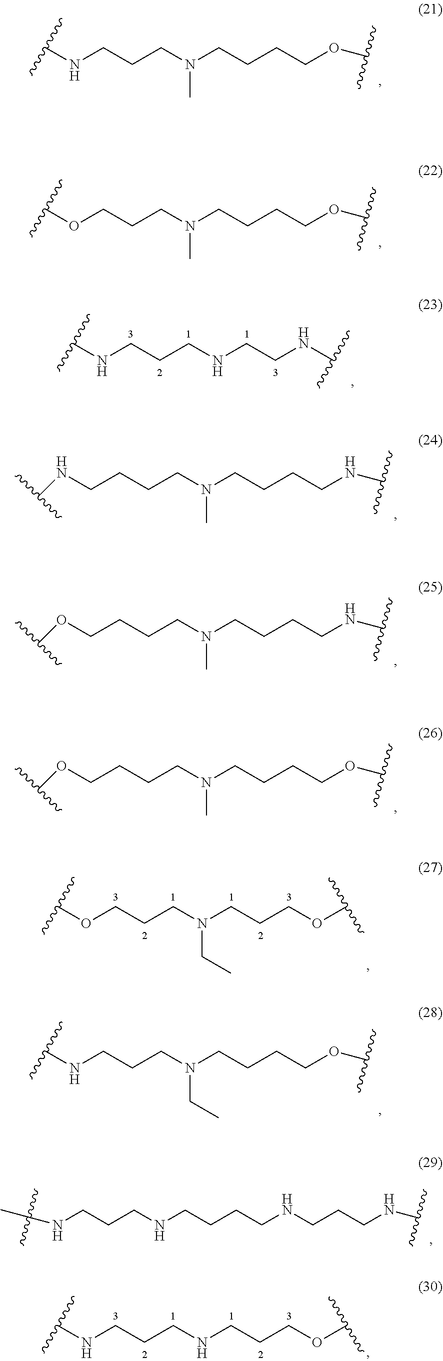

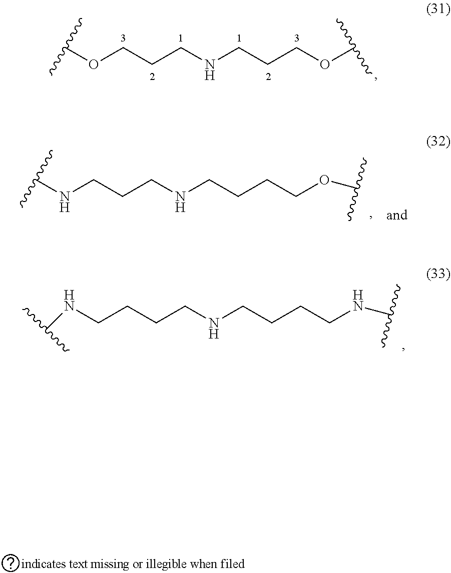 [SMILES: *OCCCNCCCO* ; *NCCCNCCCCO* ; *NCCCCNCCCCN*]

(vii) B is a C₁-C₂₂ alkyl or C₂-C₂₂ alkenyl group, preferably, an alkenyl group, in which the alkyl or alkenyl group is substituted with one to four substituents selected from the group consisting of: halogen, —CN, —NO_{3/4}, —N₃, C₁-C₆ alkyl, halo(C₁-C₆ alkyl), —OR, —NR₂, —CO₂R, —OC(O)R, —CON(R)₂, —OC(O)N(R)₂, —NHC(O)N(R)₂, —NHC(NH)N(R)_{3/4}, C₃-C₈ cycloalkyl, C₃-C₈ cycloalkenyl, aryl, heteroaryl, and heterocycle, and R is selected from the group consisting of: hydrogen, C₁-C₆ alkyl, halo(C₁-C₆ alkyl), C₃-C₈ cycloalkyl, C₃-C₈ cycloalkenyl, aryl, heteroaryl, and heterocycle; preferred substituents are OR, —NR₂, —CO₂R, —OC(O)R, —CON(R)₂, —OC(O)N(R)₂, —NHC(O)N(R)₂, or —NHC(NH)N(R)₂; further, each cycloalkyl, cycloalkenyl, aryl, heteroaryl, and heterocycle is further substituted with R and R is independently selected from the group consisting of: halogen, —CN, —NO₂, —N₃, C₁-C₆ alkyl, and halo(C₁-C₆ alkyl); more preferably B is selected from the group consisting of: methyl, ethyl, propyl, butyl, pentyl, hexyl, heptyl, octyl, decyl, dodecyl, tridecyl, tetradecyl, pentadecyl, hexadecyl, heptadecyl, octadecyl, but-3-en-1-yl, oct-7-en-1-yl, 12-tridecenyl, 14-pentadecenyl, 17-octadecenyl, oleyl, linoleyl, and arachidoneyl; or B is derived from a fatty acid or derivative thereof such as those selected from the group consisting of: caprylic acid, capric acid, lauric acid, myristic acid, palmitic acid, stearic acid, oleic acid, linoleic acid, linolenic acid, arachidonic acid, eicosapentanoic acid, 12-hydroxy-9-cis-octadecenoic acid, 12-methyltetradecanoic acid, 12-methyltri-decanoic acid, 14-methylhexadecanoic acid, 14-methylhexadecanoic acid, 18-methyl-nonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-a-hydroxystearic acid, DL-6-hydroxylauric acid, DL-6-hydroxymyristic acid, and DL-6-hydroxypalmitic acid; optionally the fatty acid comprises one or more stable isotopes such as a stable isotope of carbon (e.g., ¹³C) and hydrogen (e.g., ²H) having the examples of octanoic acid-1-¹³C, octanoic acid-8-¹³C, octanoic acid-8,8,8-d3, octanoic-²H15 acid, decanoic acid-1-¹³C, decanoic acid-10-¹³C, decanoic-10,10,10-d3 acid, decanoic-d19 acid, undecanoic acid-1-¹³C, lauric acid-12,12,12-²H3, lauric-²H23 acid, lauric acid-1-¹³C, lauric acid-1, 12-¹³C2, tridecanoic-2,2-²H2 acid, myristic acid-14-¹³C, myristic acid-1-¹³C, myristic acid-14,14,14-²H3, myristic-²H27 acid, palmitic acid-1-¹³C, palmitic acid-16-¹³C, palmitic acid-16-¹³C, 16,16,16-²H3, palmitic acid-2H31, stearic acid-1-¹³C, stearic acid-18-¹³C, stearic acid-18,18,18-²H3, stearic-²H35 acid, oleic acid-1-¹³C, oleic acid-²H34, linolenic acid-1-¹³C, linoleic acid-²H32, arachidonic-5,6,8,9, 11,12,14,15-²H8 acid, and eicosanoic-²H39 acid.

The carrier can further contain an amine group or an amine linker. The amine group preferably is a secondary or tertiary amine group. As used herein, an “amine linker” is an amine-containing linker that connects the hydrophobic unit with a terminal chemical group present on a dendrimer or dendron surface. Amines present in an amine linker may include functional groups that contain a basic nitrogen atom with a lone pair. Amines are formally derivatives of ammonia, wherein one or more hydrogen atoms have been replaced by a substituent such as a C₁-C₂₀ (e.g., C₁-C₁₀ and C₁-C₆) alkyl group and a C₂-C₂₀ (e.g., C₂-C₁₀ and C₂-C₆) alkenyl group. The amine linker may include a moiety that imparts proton-accepting functionality to a carrier by containing one or more nitrogen atoms with lone pairs. Amine linker may thus be able to accept a free proton (H′) under acidic conditions. A nitrogen atom in an amine linker may be present in the form of a secondary amine. A nitrogen atom in an amine linker may be present in the form of a tertiary amine. An amine linker may be derived, without limitation, from N1-(2-aminoethyl)ethane-1,2-diamine, N1-(2-amino-ethyl)propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-aminoethyl)ethane-1,2-diamine), N1-(2-(4-(2-aminoethyl)piperazin-1-yl)ethyl)ethane-1,2-diamine, N1-(2-amino-ethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl)(methyl)amino)-propan-1-ol, 3,3′-(methylazanediyl)bis(propan-1-ol), N1-(3-aminopropyl)-N1-methylbutane-1,4-diamine, 4-((3-aminopropyl)(methyl)amino)butan-1-ol, 4-((3-hydroxypropyl)(methyl)-amino)butan-1-ol, 4-((3-hydroxypropyl)(methyl)amino)butan-1-ol, N1-(4-aminobutyl)-N1-methylbutane-1,4-diamine, 4-((4-aminobutyl)(methyl)amino)butan-1-ol, 4,4′-(methylazane-diyl)bis(butan-1-ol), 3-((3-aminopropyl)(ethyl)amino)propan-1-ol, 3,3′-(ethylazanediyl)-bis(propan-1-ol), N1-(3-aminopropyl)-N1-ethylbutane-1,4-diamine, 4-((3-aminopropyl)-(ethyl)amino)butan-1-ol, 4-(ethyl(3-hydroxypropyl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl)bis(butan-1-ol), 3-((3-aminopropyl)amino)propan-1-ol, N1-(3-amino-propyl)butane-1,4-diamine, 4-((3-hydroxypropyl)amino)butan-1-ol, N1-(4-aminobutyl)-butane-1,4-diamine, 3,3′-azanediylbis(propan-1-ol), 4-((3-aminopropyl)amino)butan-1-ol, 4,4′-azanediylbis(butan-1-ol), or N1,N1′-(butane-1,4-diyl)bis(propane-1,3-diamine).

In addition, the carrier can also include a hydrophobic unit. As used herein, a “hydrophobic unit” is a group formed exclusively by hydrophobic atoms and not surrounded by water molecules. A hydrophobic unit typically includes a C₁-C₂₈ (e.g., C₁-C₂₀, C₁-C₁₅, C₁-C₁₀, and C₁-C₆) alkyl group or a C₂-C₂₈ (e.g., C₂-C₂₀, C₂-C₁₅, C₂-C₁₀, and C₂-C₆) alkenyl group. Chain length may be used to control the hydrophobicity and self-assembly properties of a carrier. The hydrophobic unit is optionally substituted with one or more (e.g., 1, 2, 3, and 4) substituents selected from halogen, —CN, —NO₂, —N₃, C₁-C₆ alkyl, halo(C₁-C₆ alkyl), —OR, —NR₂, —CO₂R, —OC(O)R, —CON(R)₂, —OC(O)N(R)₂, —NHC(O)N(R)₂, —NHC(NH)N(R)₂, C₃-C₈ cycloalkyl, C₃-C₈ cycloalkenyl, aryl, heteroaryl, or heterocycle. Each R, independently, is hydrogen, C₁-C₆ alkyl, halo(C₁-C₆ alkyl), C₃-C₈ cycloalkyl, C₃-C₈ cycloalkenyl, aryl, heteroaryl, or heterocycle. Each cycloalkyl, cycloalkenyl, aryl, heteroaryl, and heterocycle may be further substituted with one or more R′, independently selected from halogen, —CN, —NO₂, —N3, C₁-C₆ alkyl, or halo(C₁-C₆ alkyl). A hydrophobic unit may be included in the carrier with a functional reagent such as a fatty acid or its derivatives. A fatty acid may be a saturated or unsaturated fatty acid having C₄-C₂₈ chains. Exemplary fatty acids include caprylic acid, capric acid, lauric acid, myristic acid, palmitic acid, stearic acid, oleic acid, linoleic acid, linolenic acid, arachidonic acid, eicosapentanoic acid. A fatty acid derivative can also be used, such as 12-hydroxy-9-cis-octadecenoic acid (ricinoleic acid), 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, or DL-β-hydroxypalmitic acid. Hydrophobic unit 112 may be a methyl, ethyl, propyl, butyl, pentyl, hexyl, heptyl, octyl, decyl, undecyl, dodecyl, tridecyl, tetradecyl, pentadecyl, hexadecyl, heptadecyl, octadecyl, but-3-en-1-yl, oct-7-en-1-yl, 12-tridecenyl, 14-pentadecenyl, 17-octadecenyl, oleyl, linoleyl, arachidoneyl, 16-hydroxyhexadecyl, and 12-hydroxy-9-cis-octadecenyl(ricinoleyl) group.

The carrier can further contain a functional group suitable for tracking the carrier i.e., a functional group suitable for tracking delivery material in vitro or in vivo. This tracking group may contain stable isotopes of carbon (C) or hydrogen (H), such as ¹³C or ²H (also referred to herein as deuterium, D or d). When a carrier is formulated into nanoparticles with nucleic acids, such as a replicon RNA, the nanoparticles may be tracked in vitro and in vivo post-administration by techniques such as mass spectroscopy or nuclear magnetic resonance imaging. Inclusion of stable isotopes may be beneficial for identification of delivery molecules since these isotopes differ from the relatively abundant ¹²C and ¹H isotopes that predominate in tissues. Tracking may be useful for identifying biodistribution, material clearance and molecular stability of nanoparticles post-administration, and related issues.

Provided below are exemplary dendrimers useful as the nucleic acid carrier. Their preparations are demonstrated WO2021207020A1 and WO 2022/212838 A1.

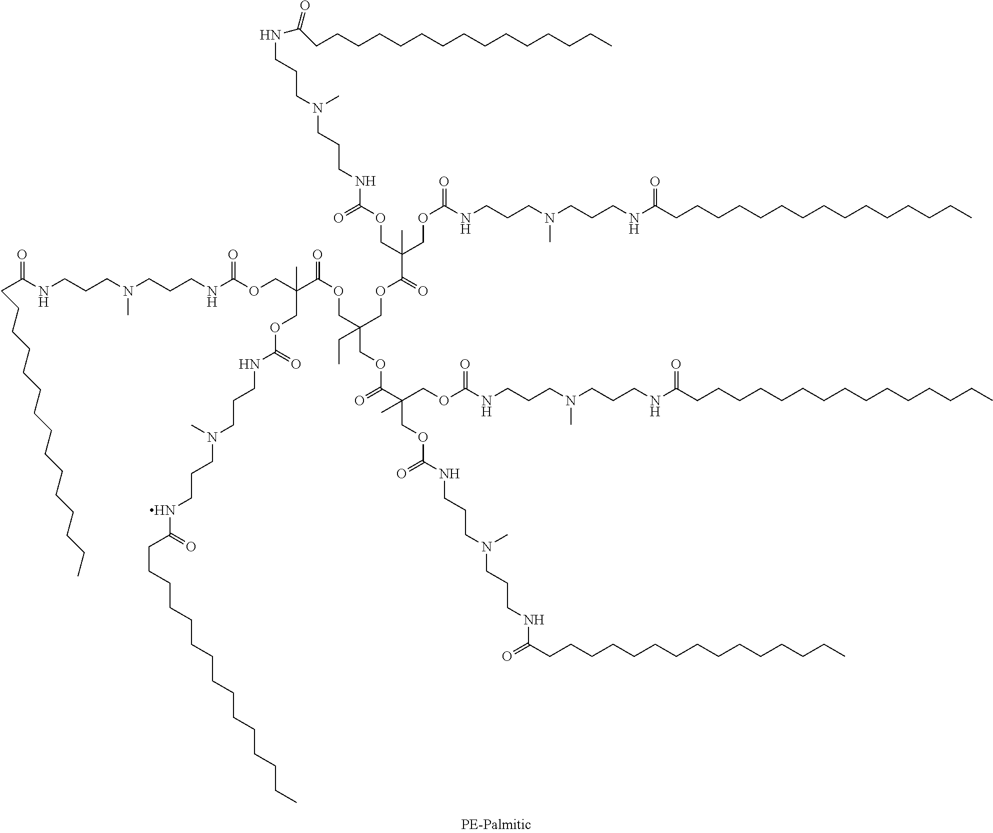

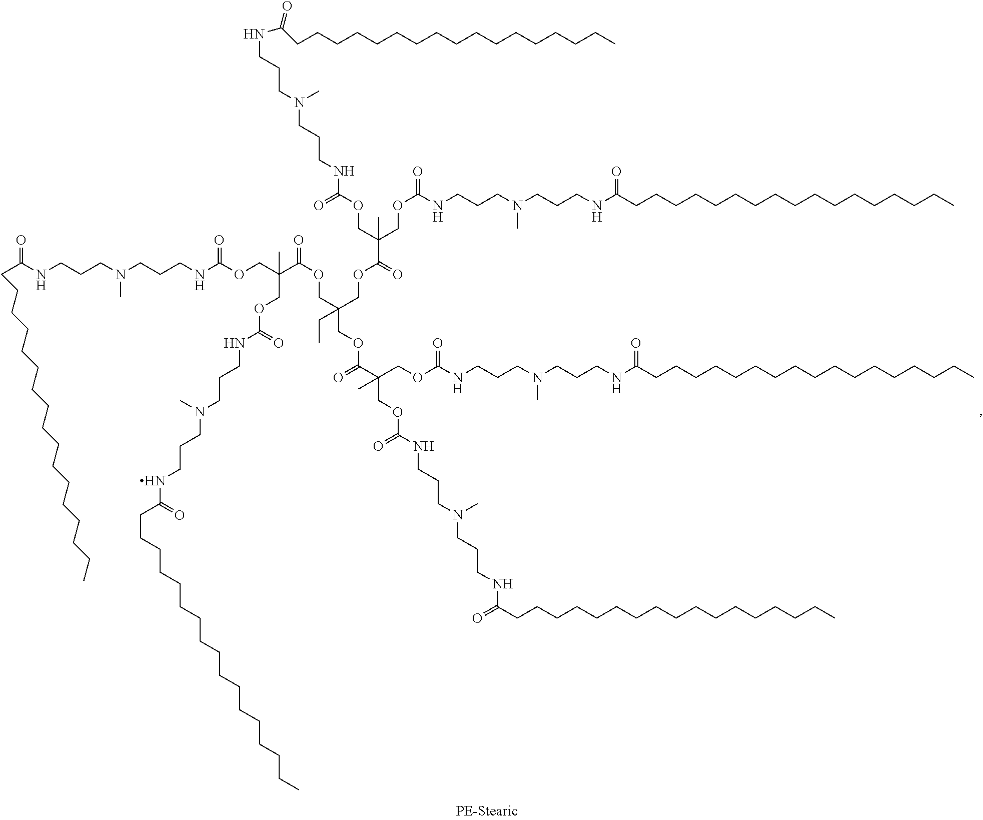

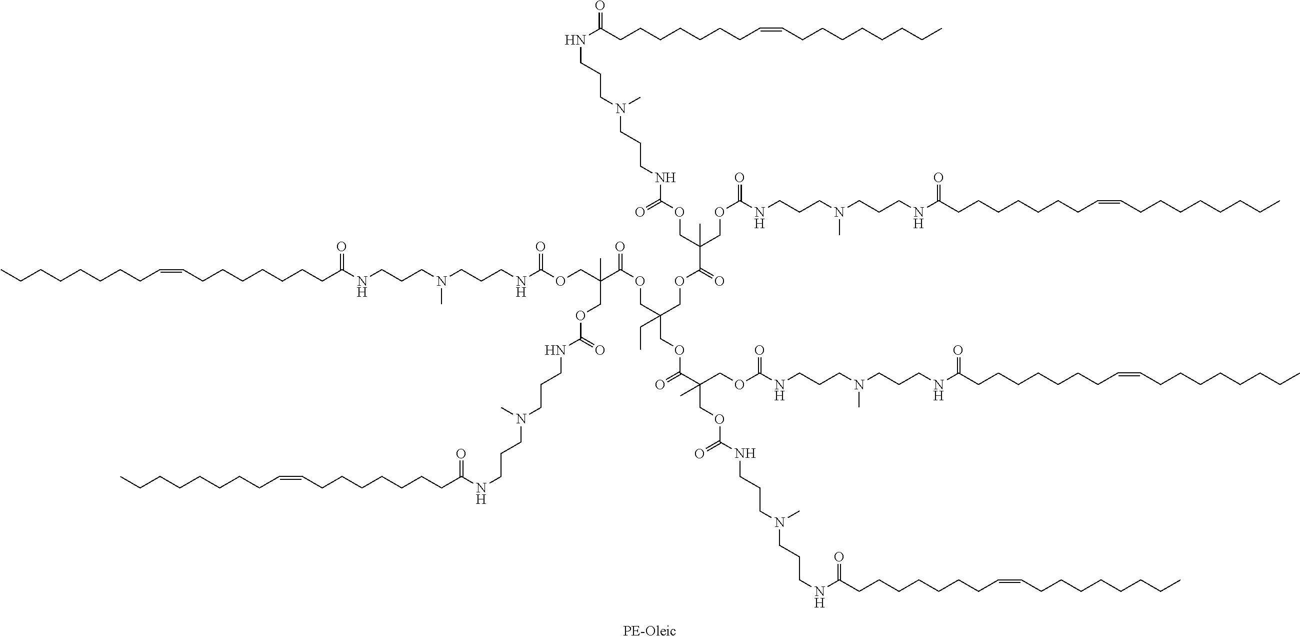 [SMILES: CCCCCCCC/C=C\CCCCCCCC(=O)NCCCN(C)CCCNC(=O)OCC(C)(COC(=O)NCCCN(C)CCCNC(=O)CCCCCCC/C=C\CCCCCCCC)C(=O)OCC(CC)(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC]

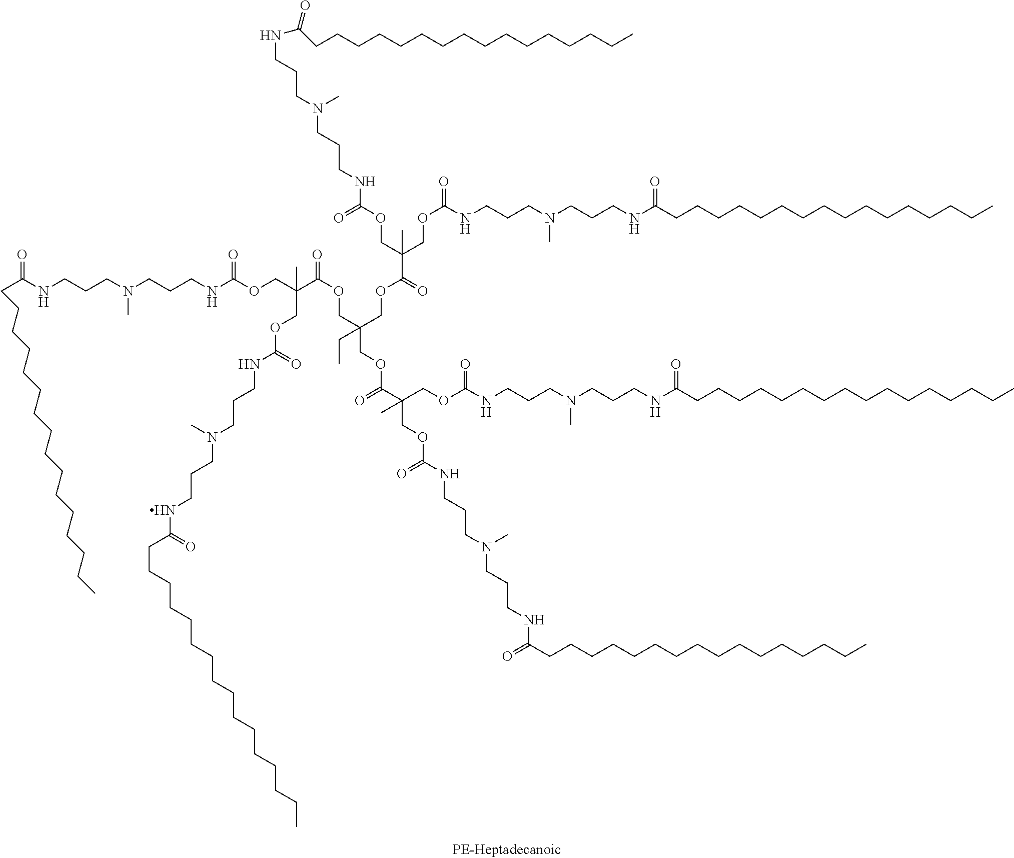

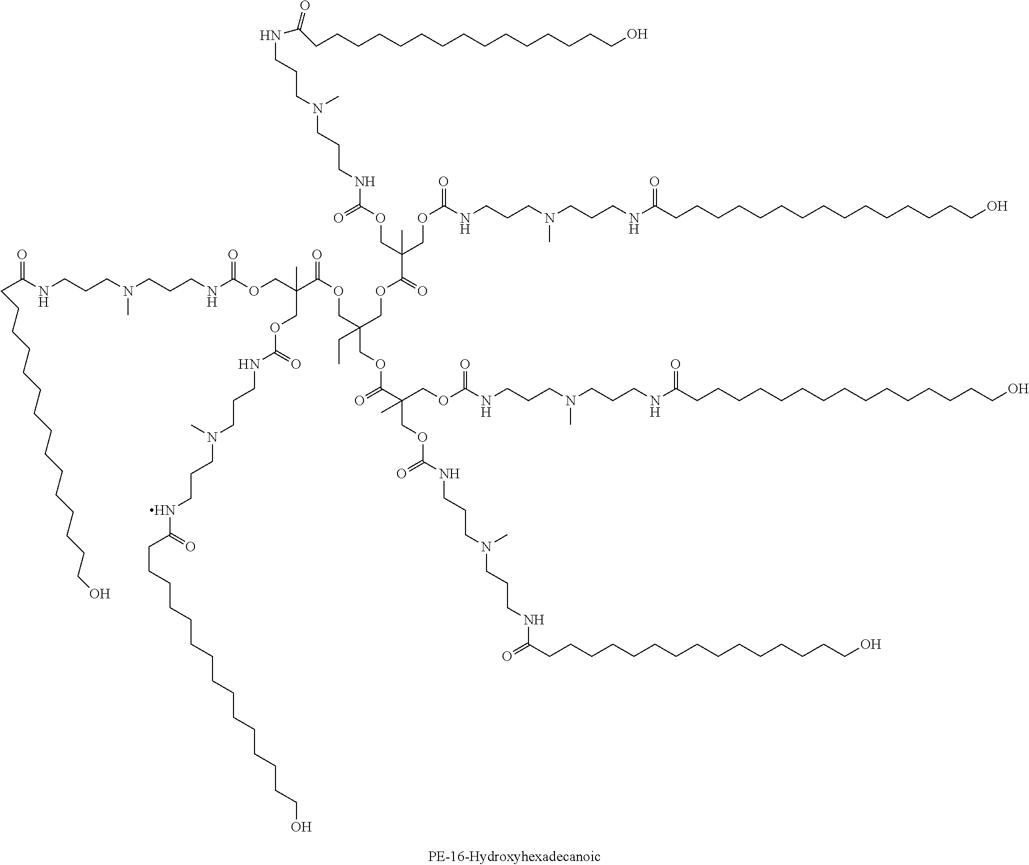

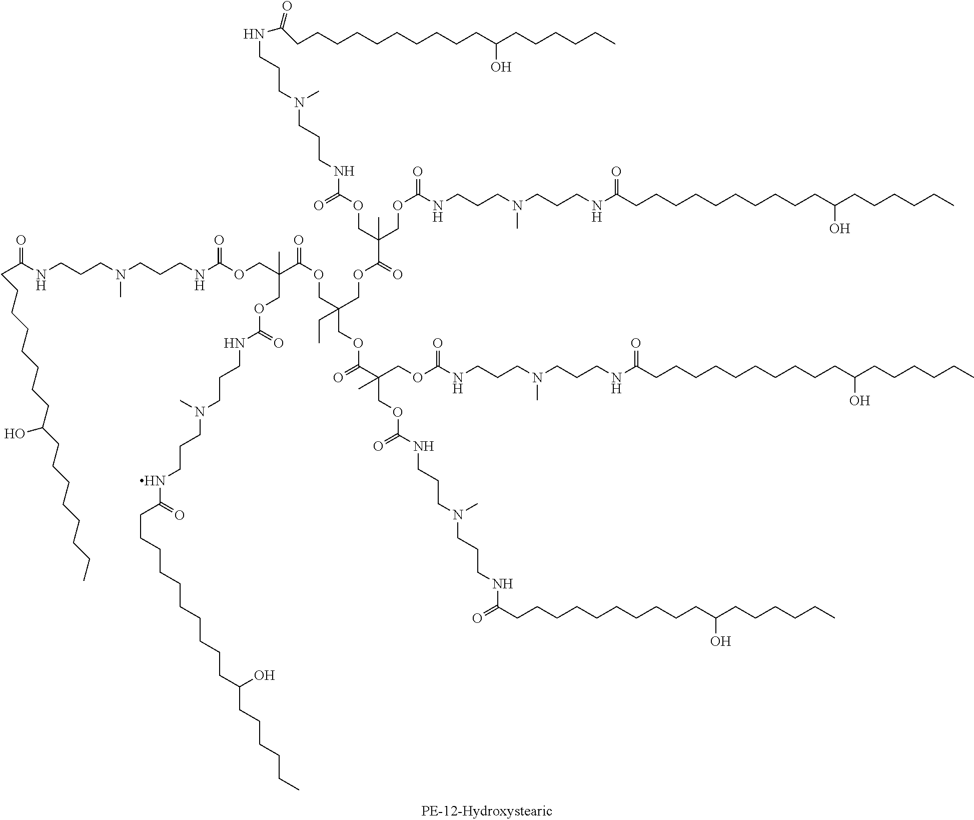

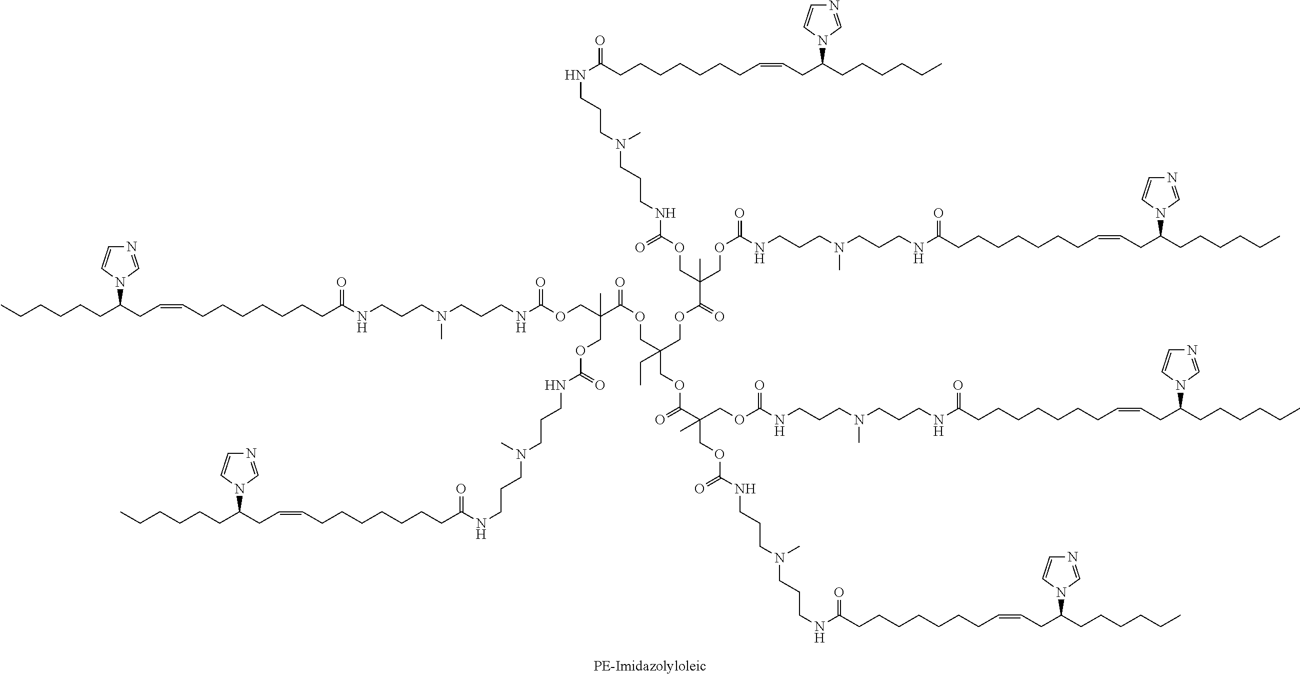 [SMILES: CCCCCC[C@@H](C/C=C\CCCCCCCC(=O)NCCCN(C)CCCNC(=O)OCC(C)(COC(=O)NCCCN(C)CCCNC(=O)CCCCCCC/C=C\C[C@H](CCCCCC)n1ccnc1)C(=O)OCC(CC)(COC(=O)C(C)(COC(=O)NCCCN(C)CCCNC(=O)CCCCCCC/C=C\C[C@H](CCCCCC)n1ccnc1)COC(=O)NCCCN(C)CCCNC(=O)CCCCCCC/C=C\C[C@H](CCCCCC)n1ccnc1)COC(=O)C(C)(COC(=O)NCCCN(C)CCCNC(=O)CCCCCCC/C=C\C[C@@H](CCCCCC)n1ccnc1)COC(=O)NCCCN(C)CCCNC(=O)CCCCCCC/C=C\C[C@@H](CCCCCC)n1ccnc1)n1ccnc1]

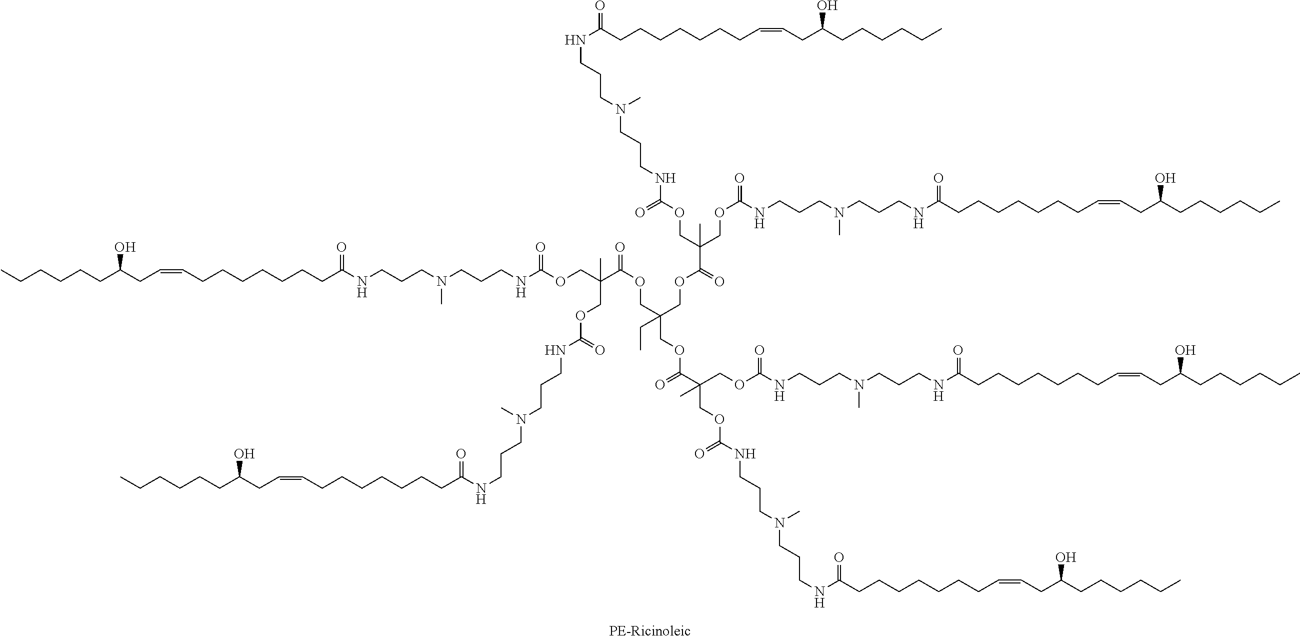 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@@H](O)CCCCCC)C(=O)OCC(CC)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)COC(=O)C(C)(COC(=O)NCCCN(C)CCCNC(=O)CCCCCCC/C=C\C[C@H](O)CCCCCC)COC(=O)NCCCN(C)CCCNC(=O)CCCCCCC/C=C\C[C@H](O)CCCCCC]

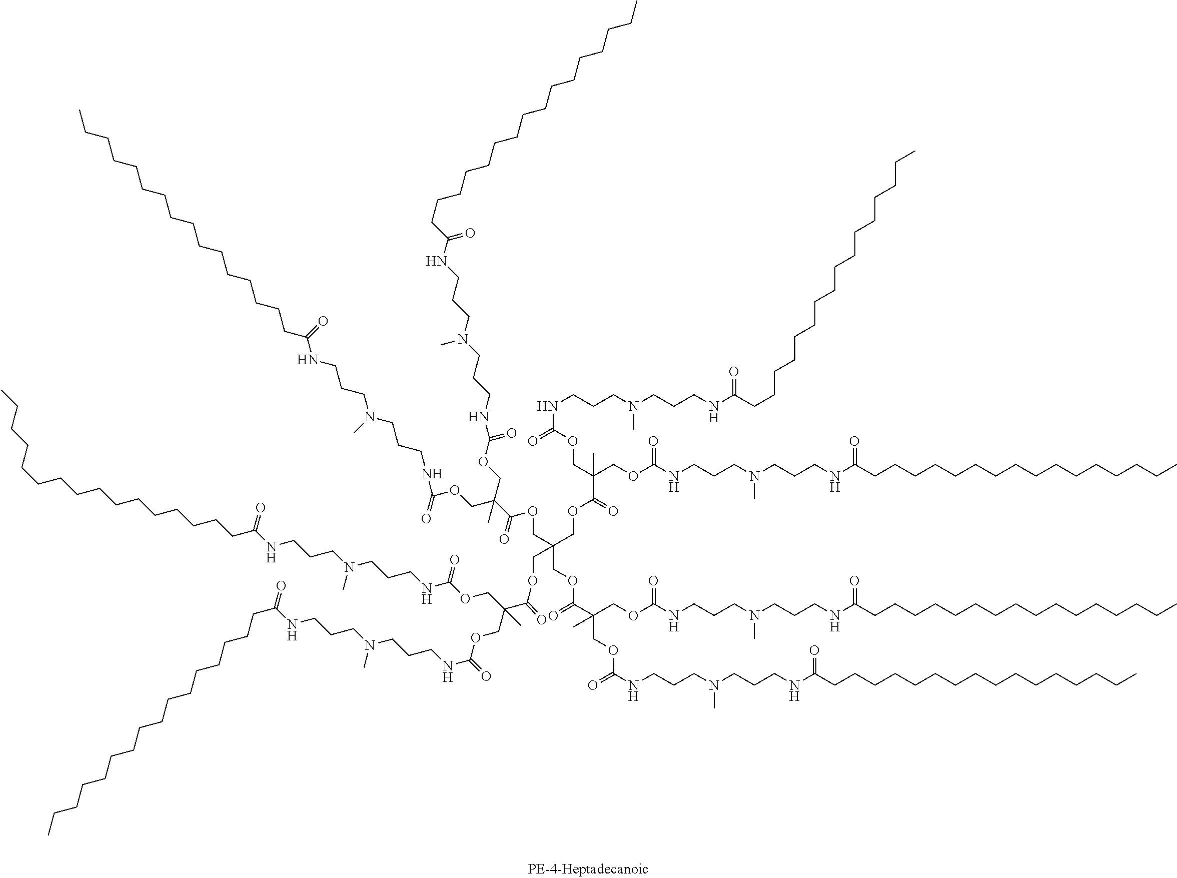 [SMILES: CCCCCCCCCCCCCCCCC(=O)NCCCN(C)CCCNC(=O)OCC(C)(COC(=O)NCCCN(C)CCCNC(=O)CCCCCCCCCCCCCCCC)C(=O)OCC(COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC)(COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC)COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC]

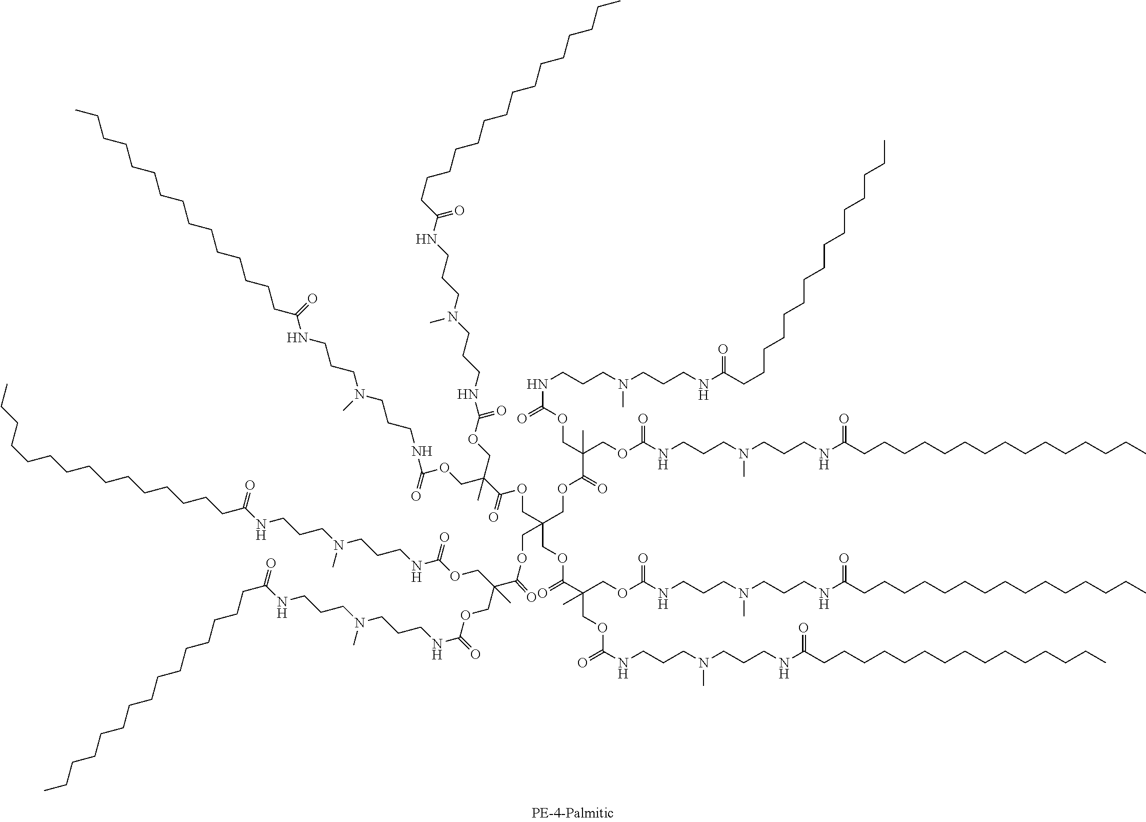 [SMILES: CCCCCCCCCCCCCCCC(=O)NCCCN(C)CCCNC(=O)OCC(C)(COC(=O)NCCCN(C)CCCNC(=O)CCCCCCCCCCCCCCC)C(=O)OCC(COC(=O)C(C)(COC(=O)NCCCN(C)CCCNC(=O)CCCCCCCCCCCCCCC)COC(=O)NCCCN(C)CCCNC(=O)CCCCCCCCCCCCCCC)(COC(=O)C(C)(COC(=O)NCCCN(C)CCCNC(=O)CCCCCCCCCCCCCCC)COC(=O)NCCCN(C)CCCNC(=O)CCCCCCCCCCCCCCC)COC(=O)C(C)(COC(=O)NCCCN(C)CCCNC(=O)CCCCCCCCCCCCCCC)COC(=O)NCCCN(C)CCCNC(=O)CCCCCCCCCCCCCCC]

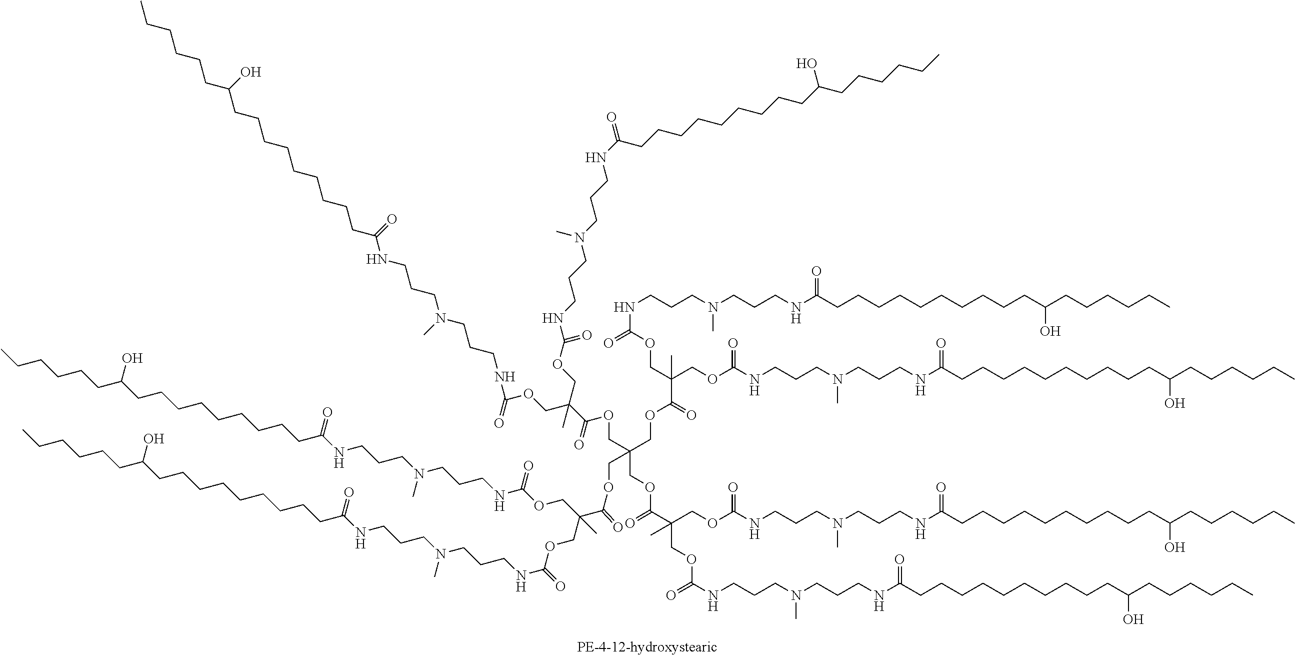 [SMILES: CCCCCCC(O)CCCCCCCCCCC(=O)NCCCN(C)CCCNC(=O)OCC(C)(COC(=O)NCCCN(C)CCCNC(=O)CCCCCCCCCCC(O)CCCCCC)C(=O)OCC(COC(=O)C(C)(COC(=O)NCCCN(C)CCCNC(=O)CCCCCCCCCCC(O)CCCCCC)COC(=O)NCCCN(C)CCCNC(=O)CCCCCCCCCCC(O)CCCCCC)(COC(=O)C(C)(COC(=O)NCCCN(C)CCCNC(=O)CCCCCCCCCC(O)CCCCCC)COC(=O)NCCCN(C)CCCNC(=O)CCCCCCCCCC(O)CCCCCC)COC(=O)C(C)(COC(=O)NCCCN(C)CCCNC(=O)CCCCCCCCCC(O)CCCCCC)COC(=O)NCCCN(C)CCCNC(=O)CCCCCCCCCC(O)CCCCCC]

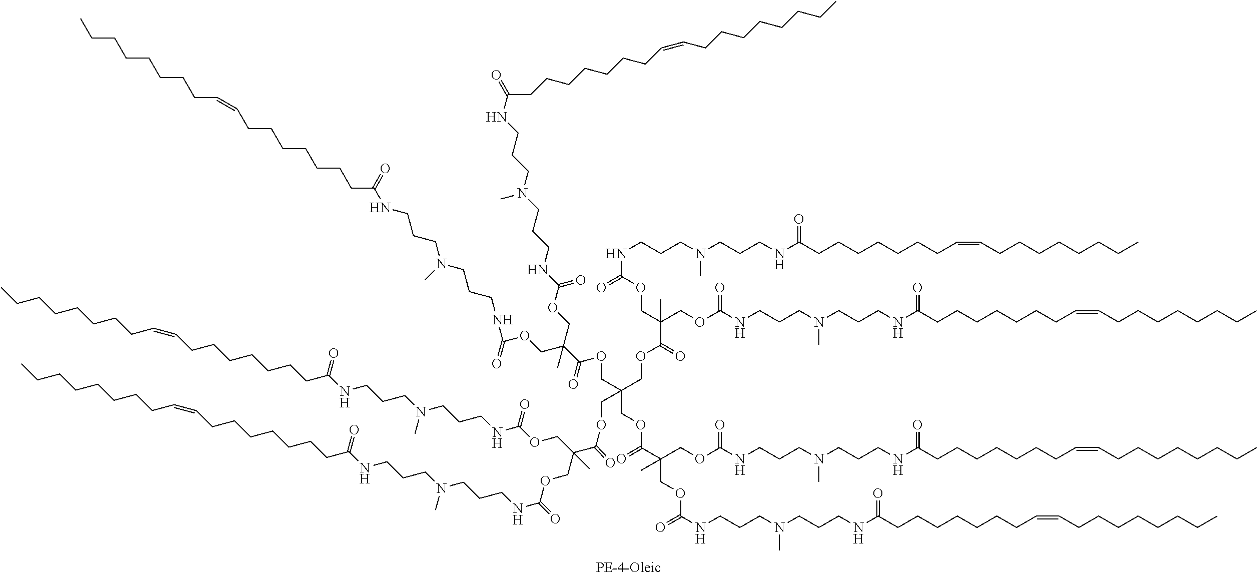 [SMILES: CCCCCCCC/C=C\CCCCCCCC(=O)NCCCN(C)CCCNC(=O)OCC(C)(COC(=O)NCCCN(C)CCCNC(=O)CCCCCCC/C=C\CCCCCCCC)C(=O)OCC(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC]

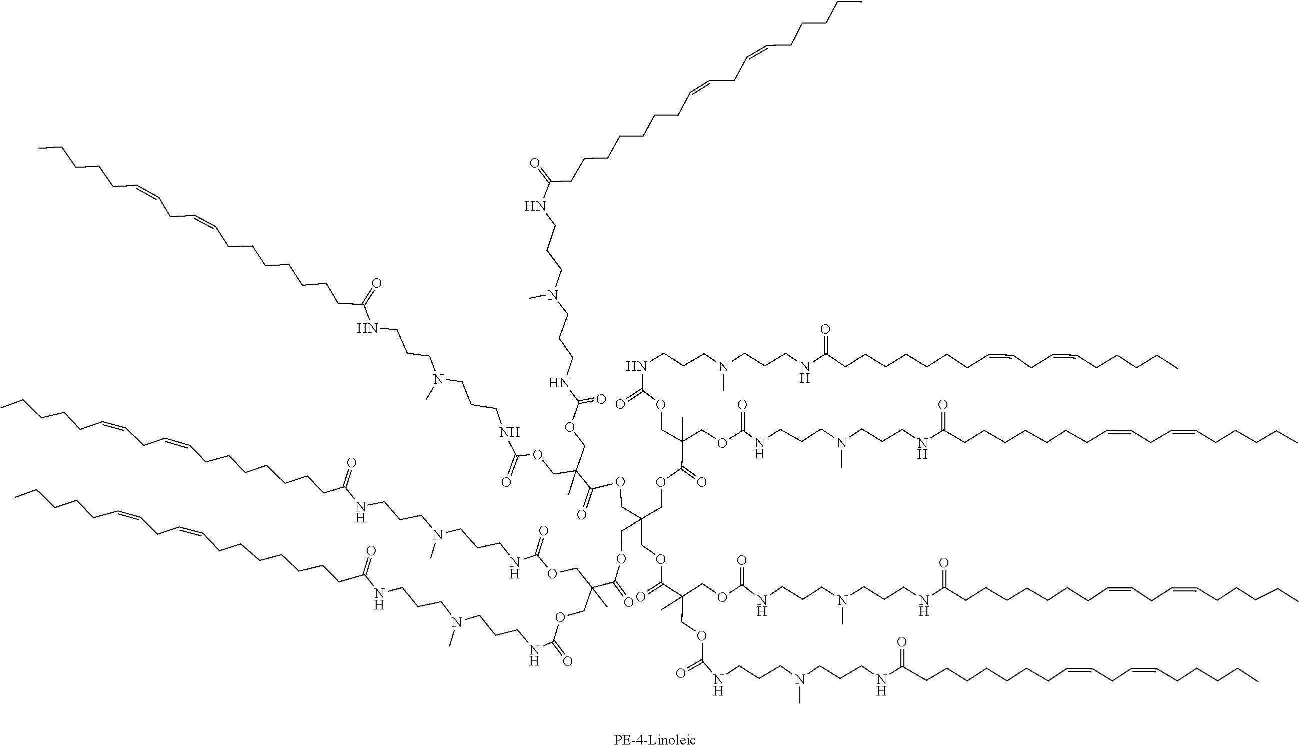 [SMILES: CCCCC/C=C\C/C=C\CCCCCCCC(=O)NCCCN(C)CCCNC(=O)OCC(C)(COC(=O)NCCCN(C)CCCNC(=O)CCCCCCC/C=C\C/C=C\CCCCC)C(=O)OCC(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC]

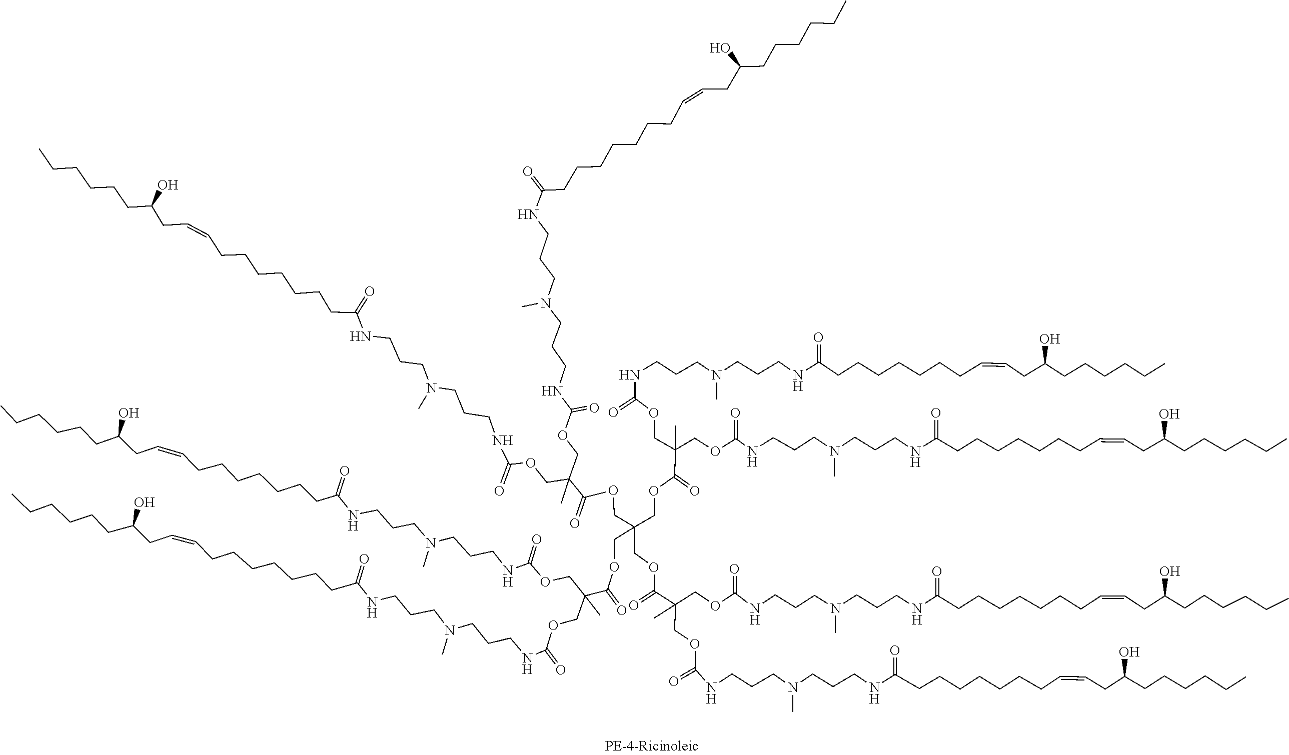 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@@H](O)CCCCCC)C(=O)OCC(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@H](O)CCCCCC)COC(=O)C(C)(COC(=O)NCCCN(C)CCCNC(=O)CCCCCCC/C=C\C[C@H](O)CCCCCC)COC(=O)NCCCN(C)CCCNC(=O)CCCCCCC/C=C\C[C@H](O)CCCCCC]

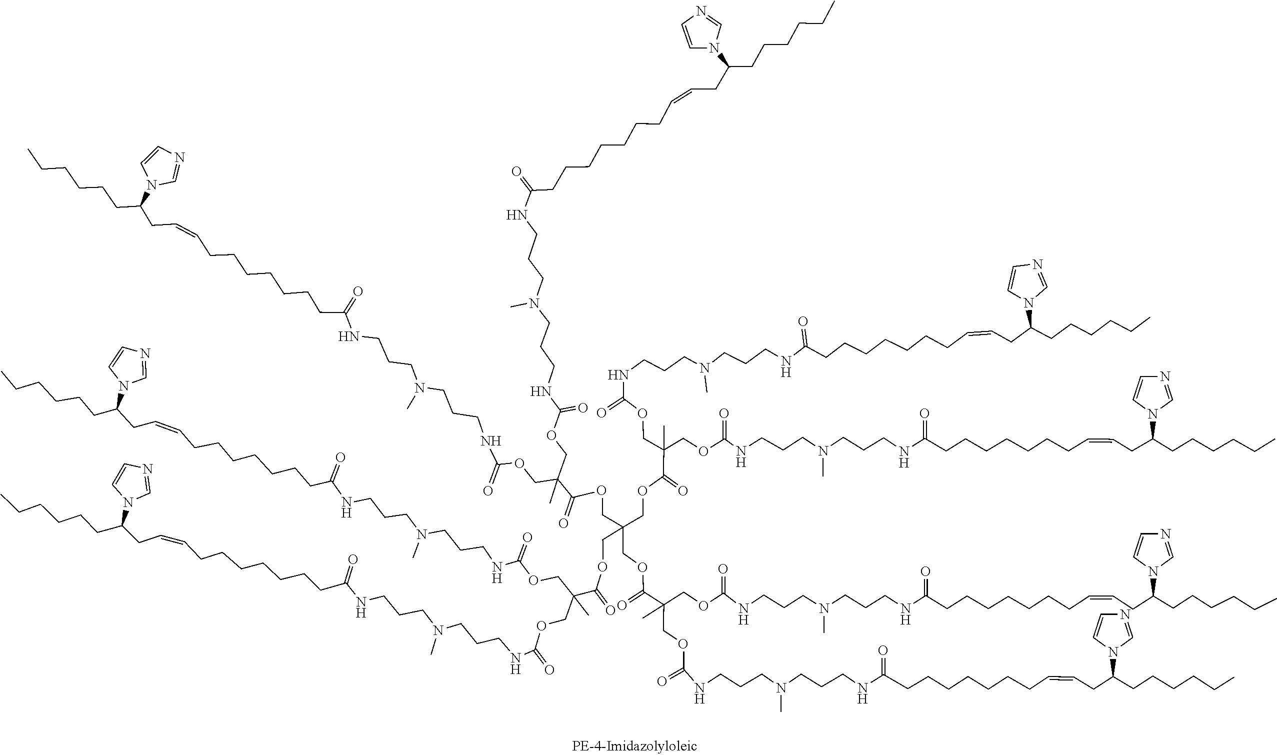

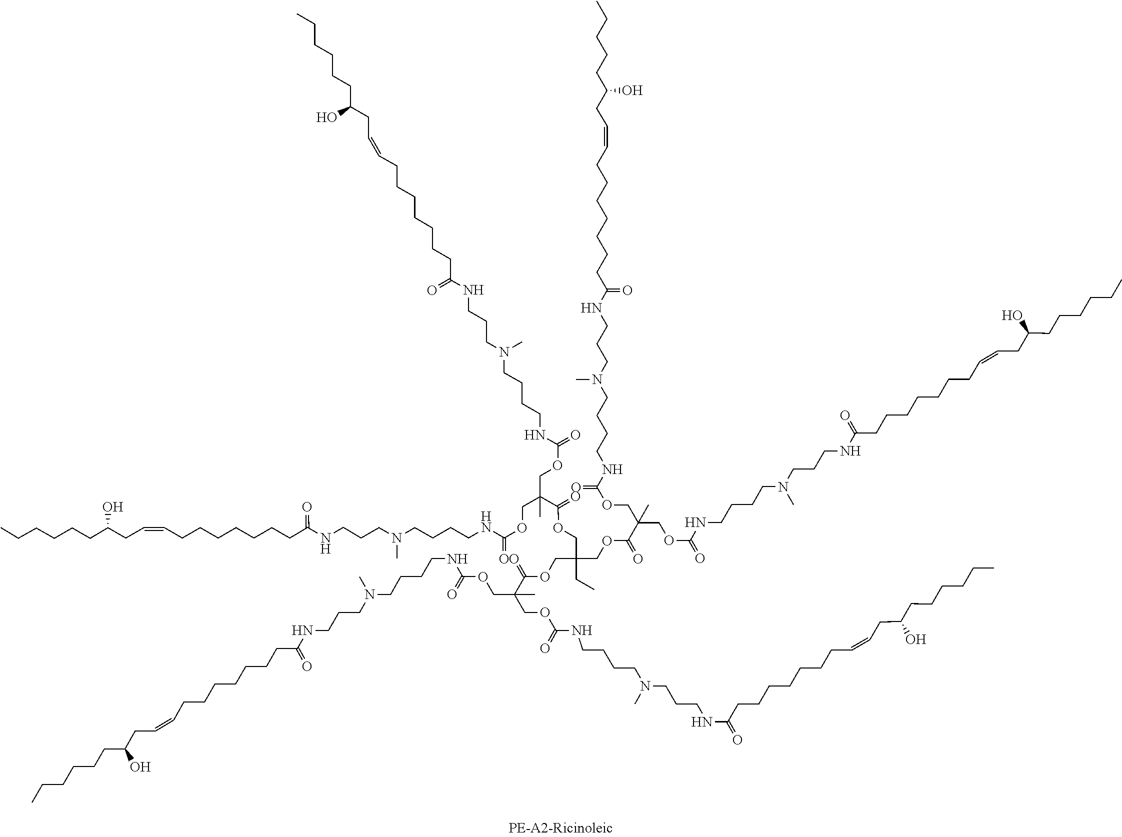 [SMILES: CCCCCC[C@H](O)C/C=C\CCCCCCCC(=O)NCCCN(C)CCCCNC(=O)OCC(C)(COC(=O)NCCCCN(C)CCCNC(=O)CCCCCCC/C=C\C[C@@H](O)CCCCC)C(=O)OCC(CC)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC]

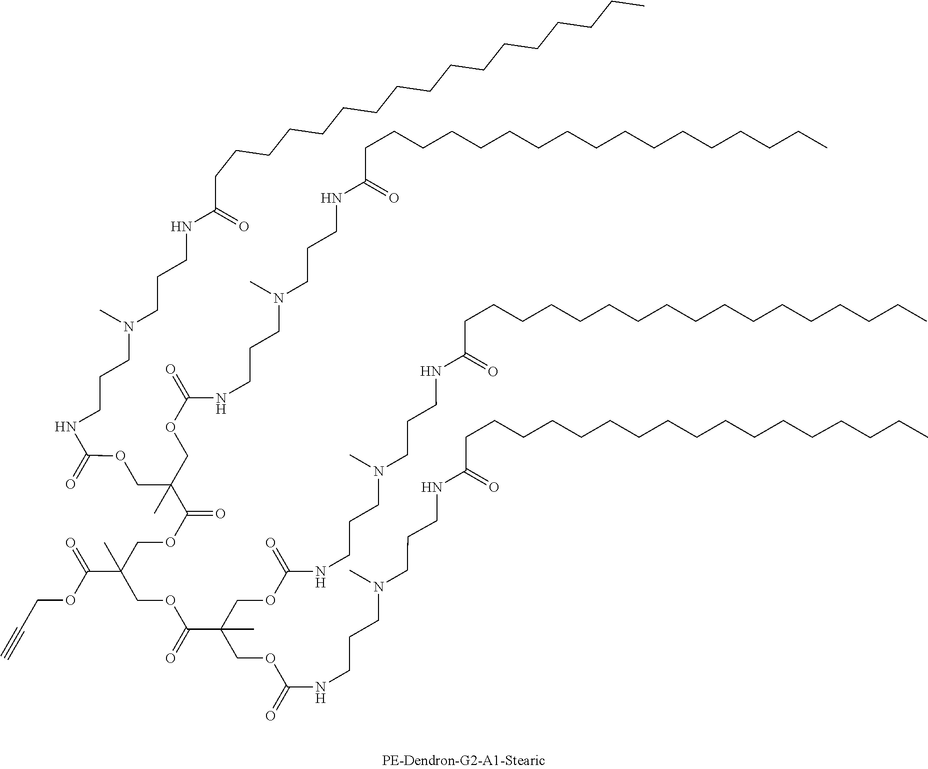 [SMILES: C#CCOC(=O)C(C)(COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC)COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC]

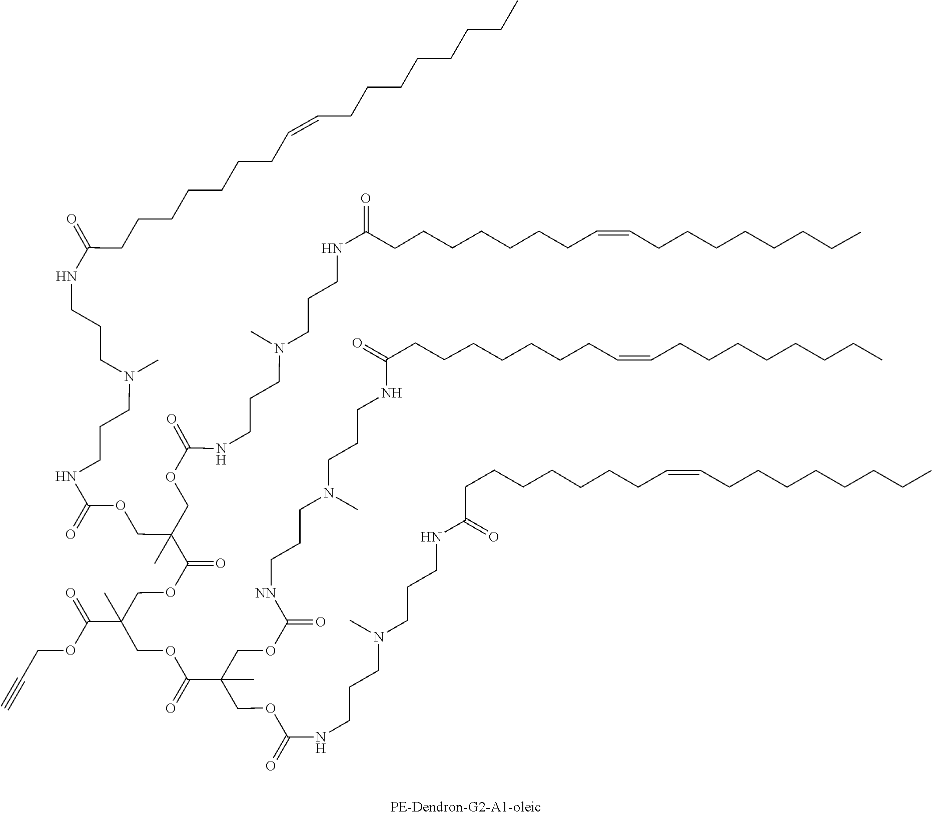

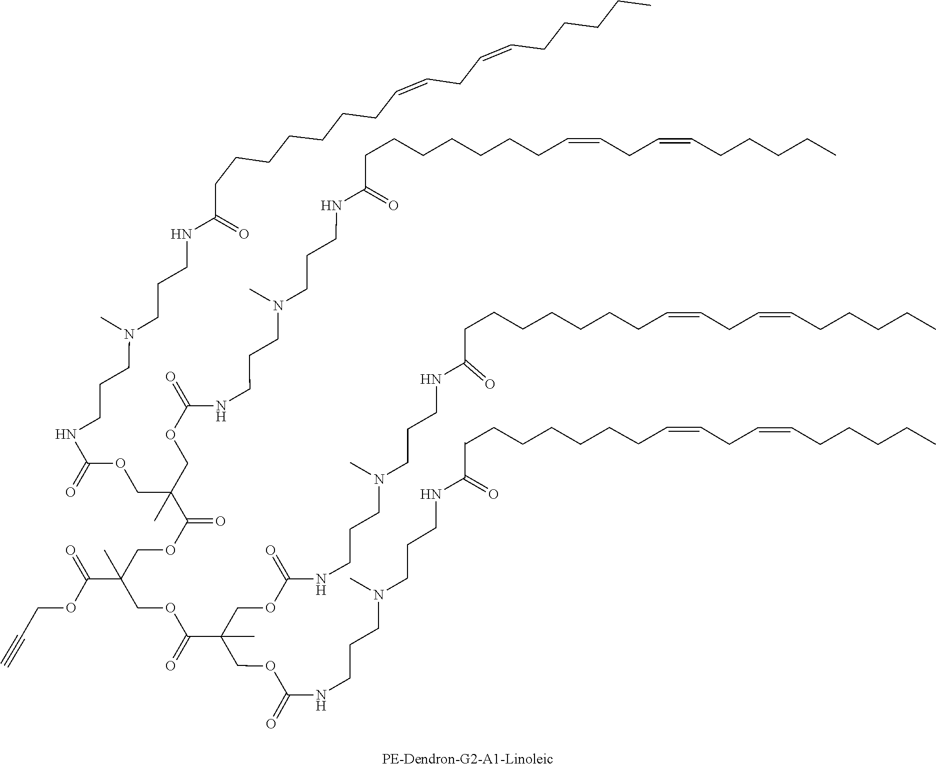 [SMILES: C#CCOC(=O)C(C)(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)COC(=O)C(C)(COC(=O)NCCCN(C)CCCNC(=O)CCCCCCC/C=C\C/C=C\CCCCC)COC(=O)NCCCN(C)CCCNC(=O)C(C)CCCCCC/C=C\C/C=C\CCCCC]

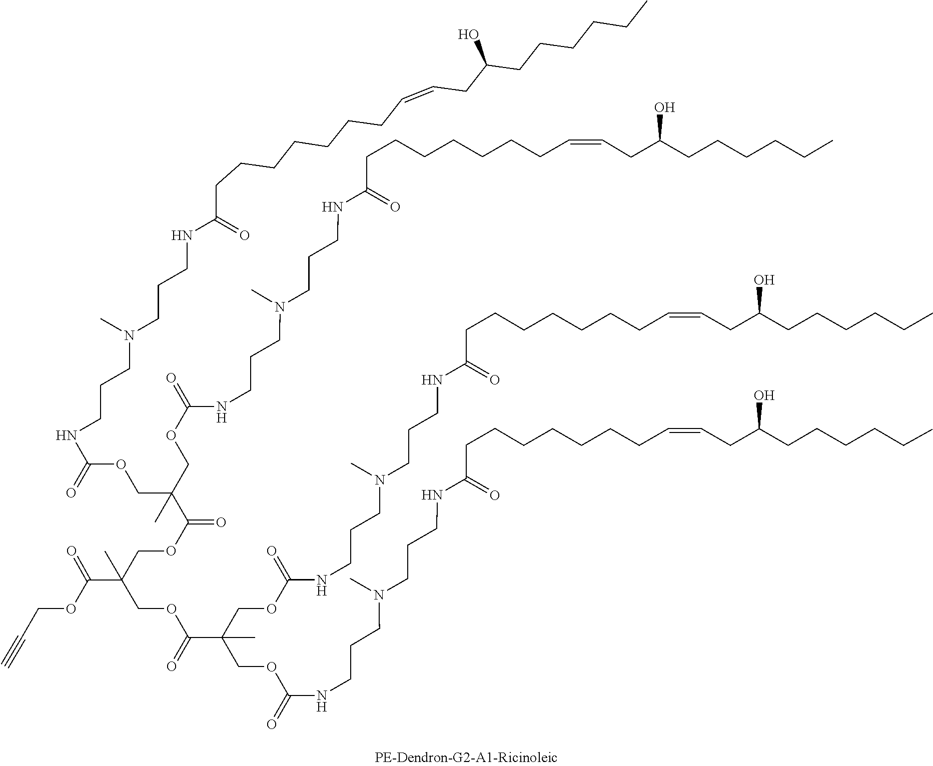 [SMILES: C#CCOC(=O)C(C)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC]

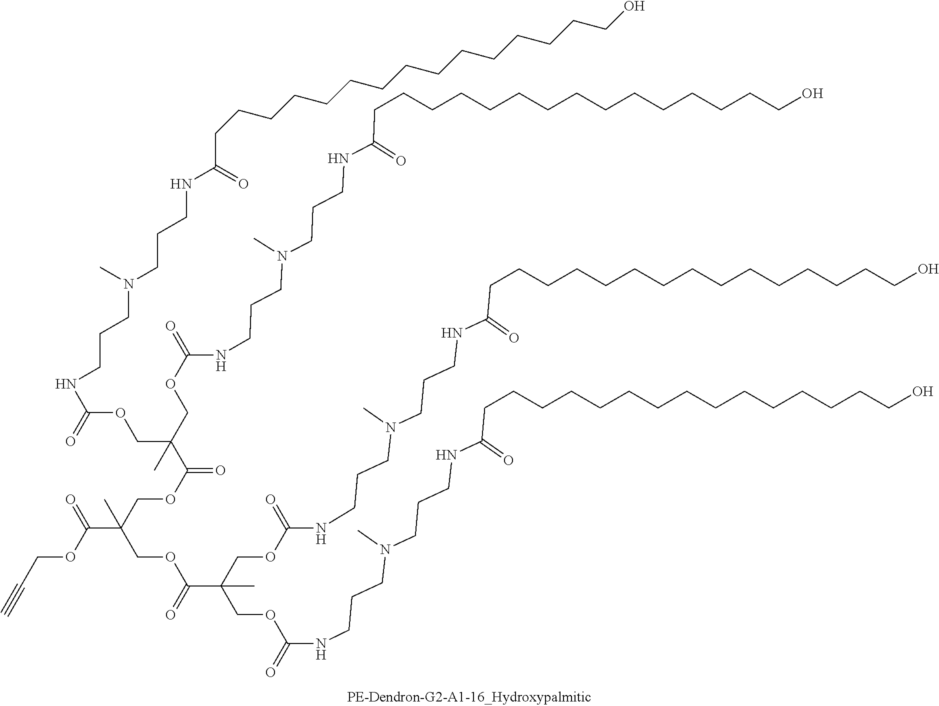 [SMILES: C#CCOC(=O)C(C)(COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO)COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO]

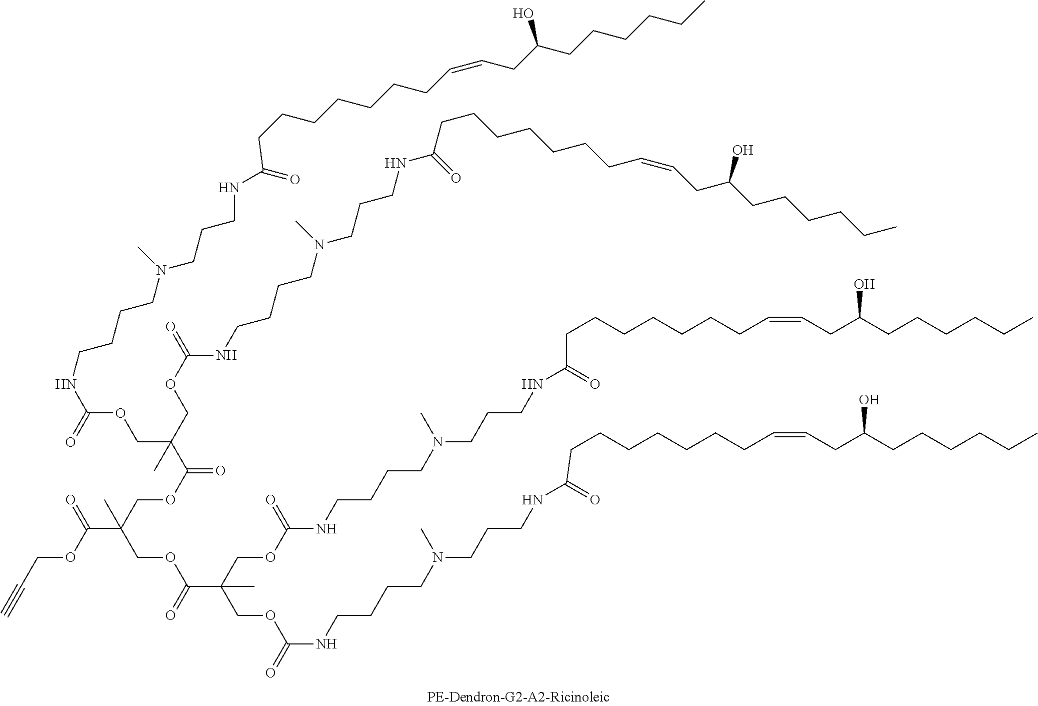 [SMILES: C#CCOC(=O)C(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC]

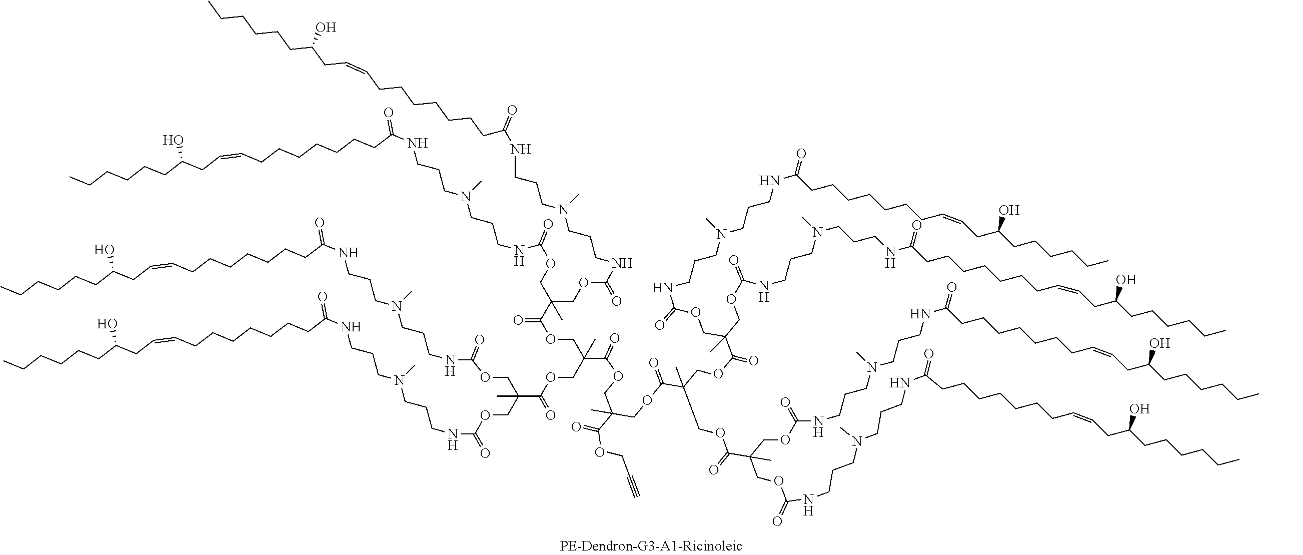

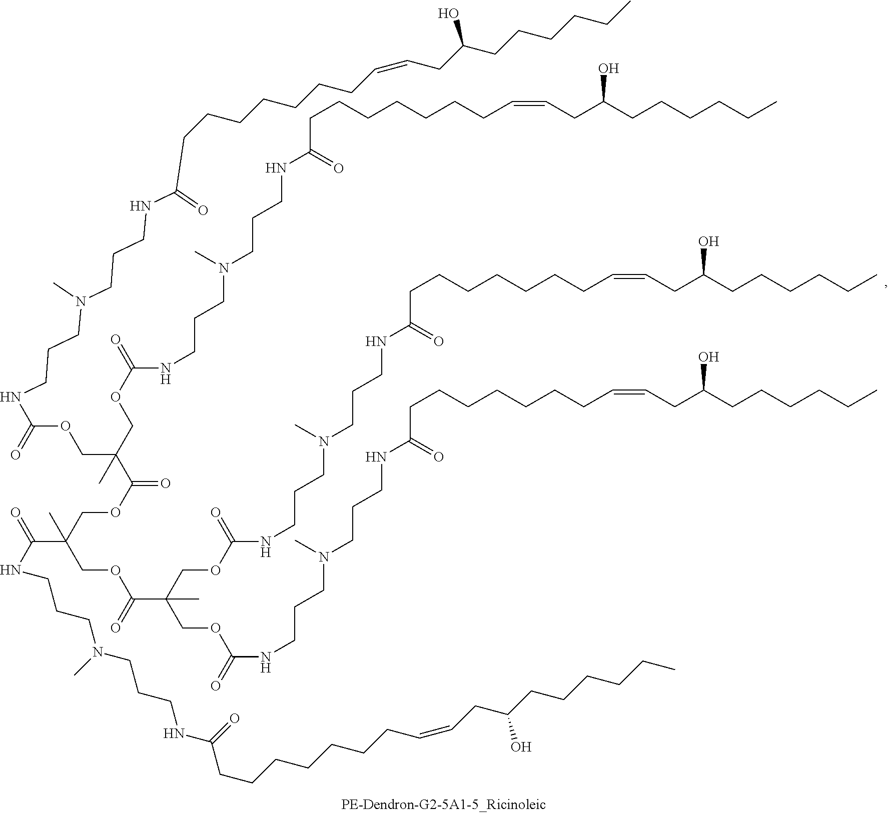 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@@H](O)CCCCCC)C(=O)OCC(C)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)C(=O)NCCCN(C)CCCNC(=O)CCCCCCC/C=C\C[C@@H](O)CCCCCC]

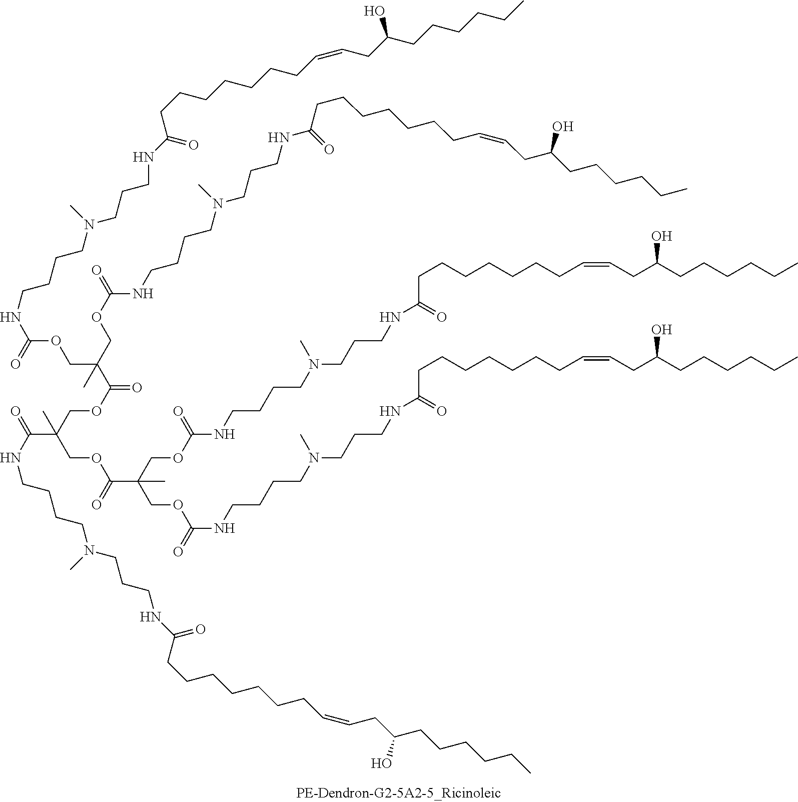 [SMILES: CCCCCC[C@H](O)C/C=C\CCCCCCCC(=O)NCCCN(C)CCCCNC(=O)OCC(C)(COC(=O)NCCCCN(C)CCCNC(=O)CCCCCCC/C=C\C[C@@H](O)CCCCCC)C(=O)OCC(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)C(=O)NCCCCN(C)CCCNC(=O)CCCCCCC/C=C\C[C@@H](O)CCCCCC]

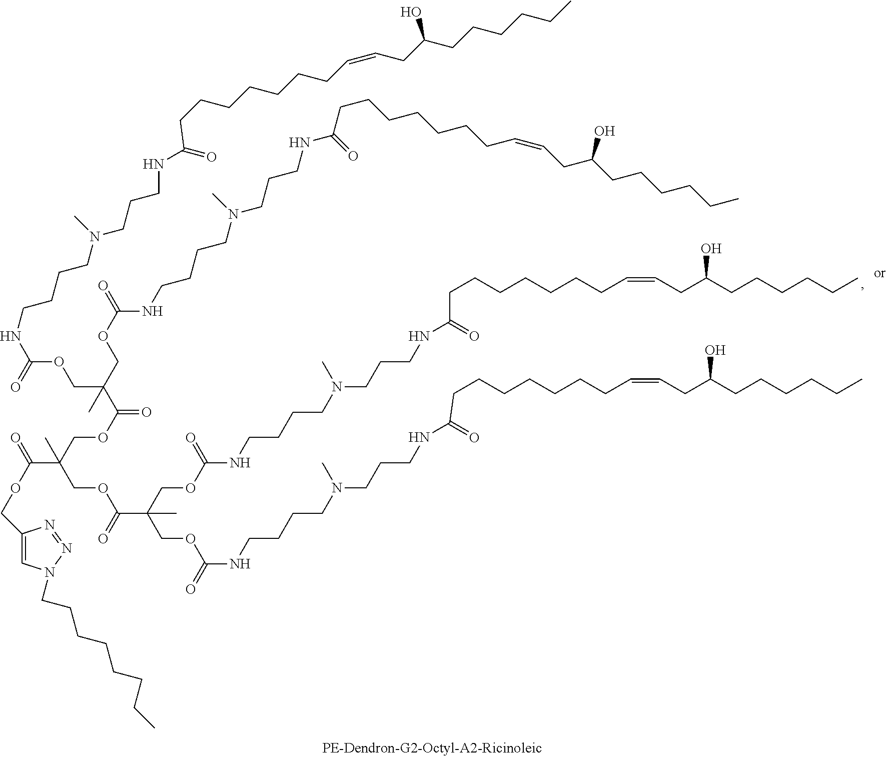 [SMILES: CCCCCCCCn1cc(COC(=O)C(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)nn1]

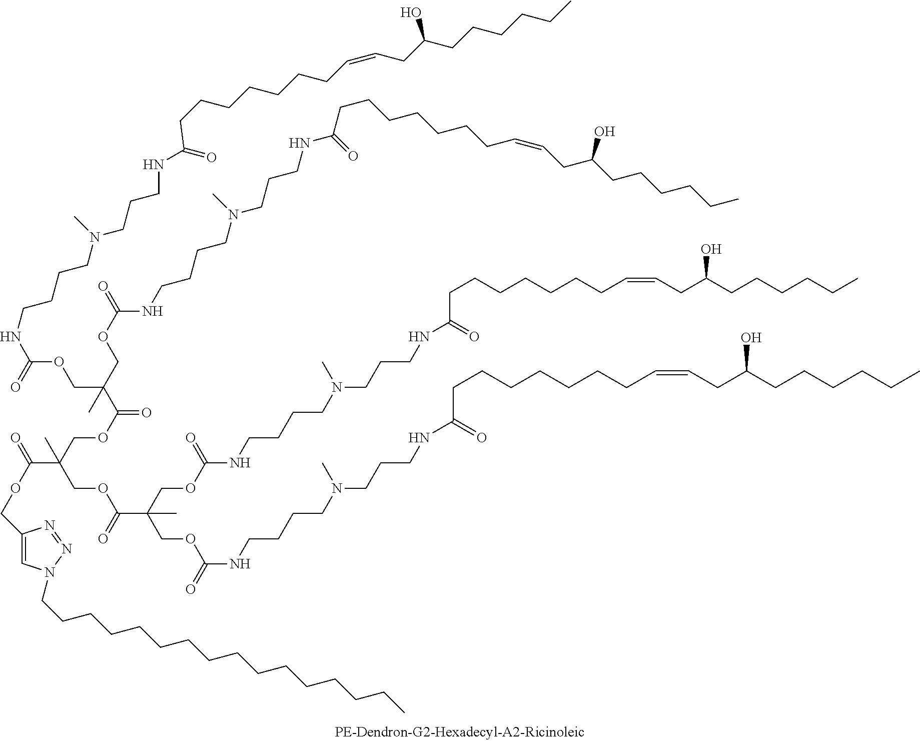 [SMILES: CCCCCCCCCCCCCCCCn1cc(COC(=O)C(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)nn1]

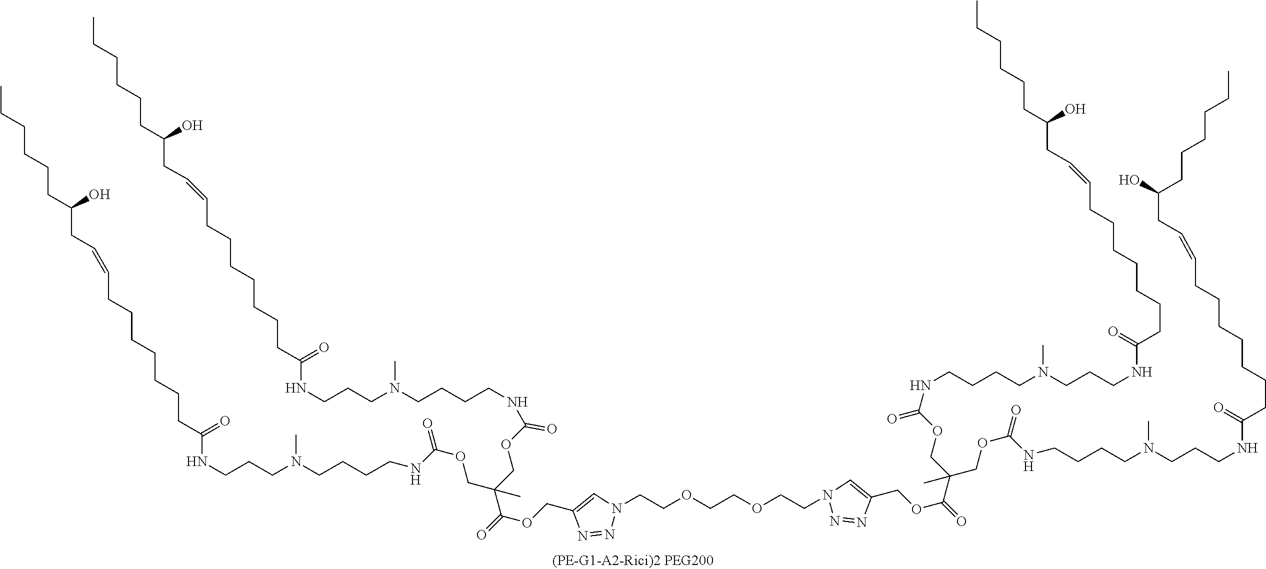 [SMILES: CCCCCC[C@H](O)C/C=C\CCCCCCCC(=O)NCCCN(C)CCCCNC(=O)OCC(C)(COC(=O)NCCCCN(C)CCCNC(=O)CCCCCCC/C=C\C[C@H](O)CCCCCC)C(=O)OCc1cn(CCOCCOCCn2cc(COC(=O)C(C)(COC(=O)NCCCCN(C)CCCNC(=O)CCCCCCC/C=C\C[C@H](O)CCCCCC)COC(=O)NCCCCN(C)CCCNC(=O)CCCCCCC/C=C\C[C@H](O)CCCCCC)nn2)nn1]

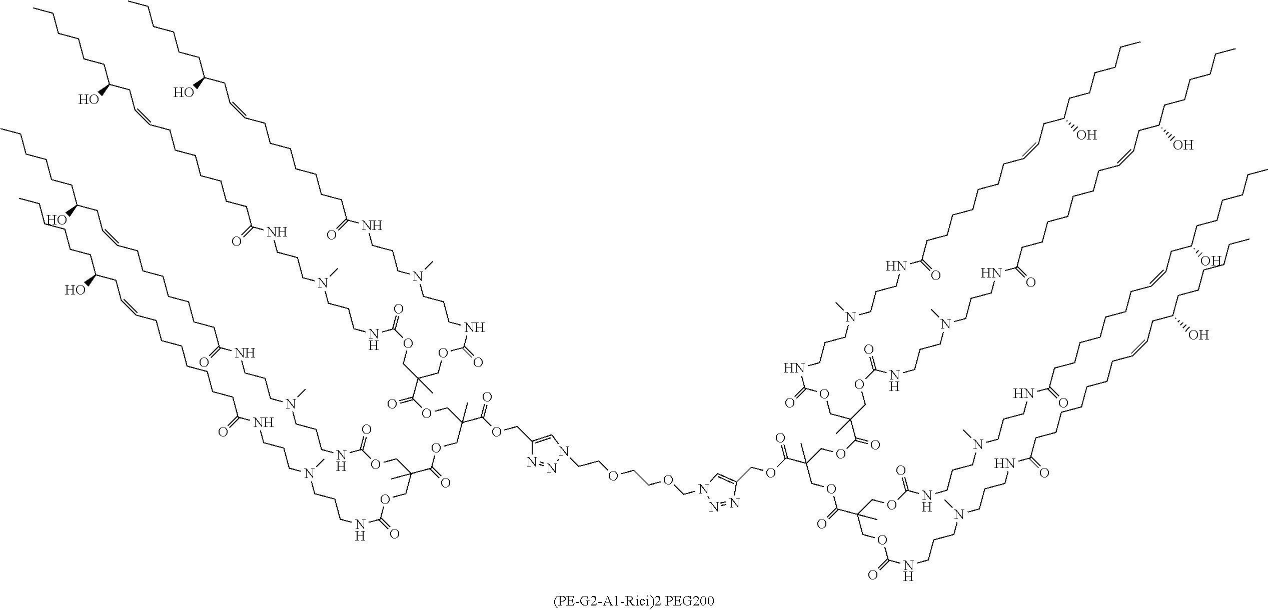

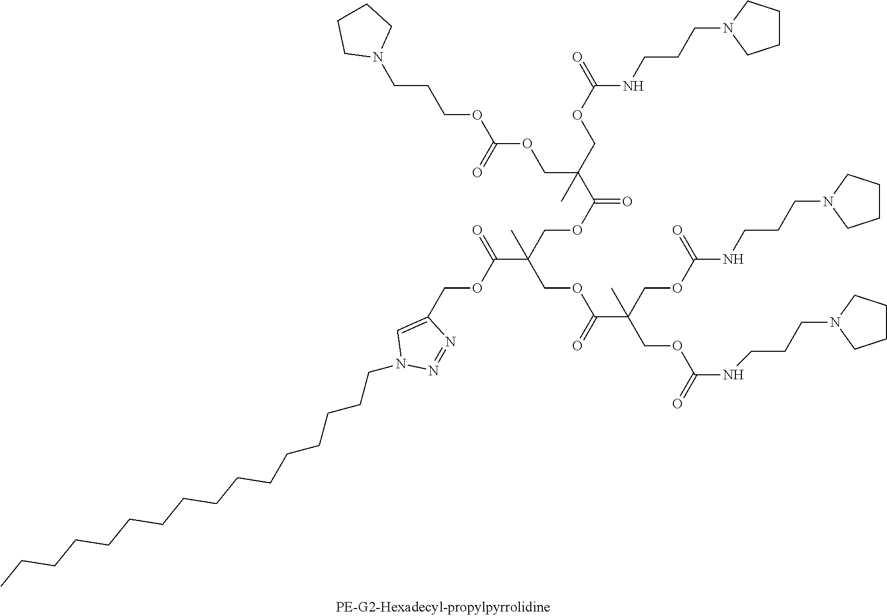 [SMILES: CCCCCCCCCCCCCCCCn1cc(COC(=O)C(C)(COC(=O)C(C)(COC(=O)NCCCN2CCCC2)COC(=O)NCCCN2CCCC2)COC(=O)C(C)(COC(=O)NCCCN2CCCC2)COC(=O)OCCCN2CCCC2)nn1]

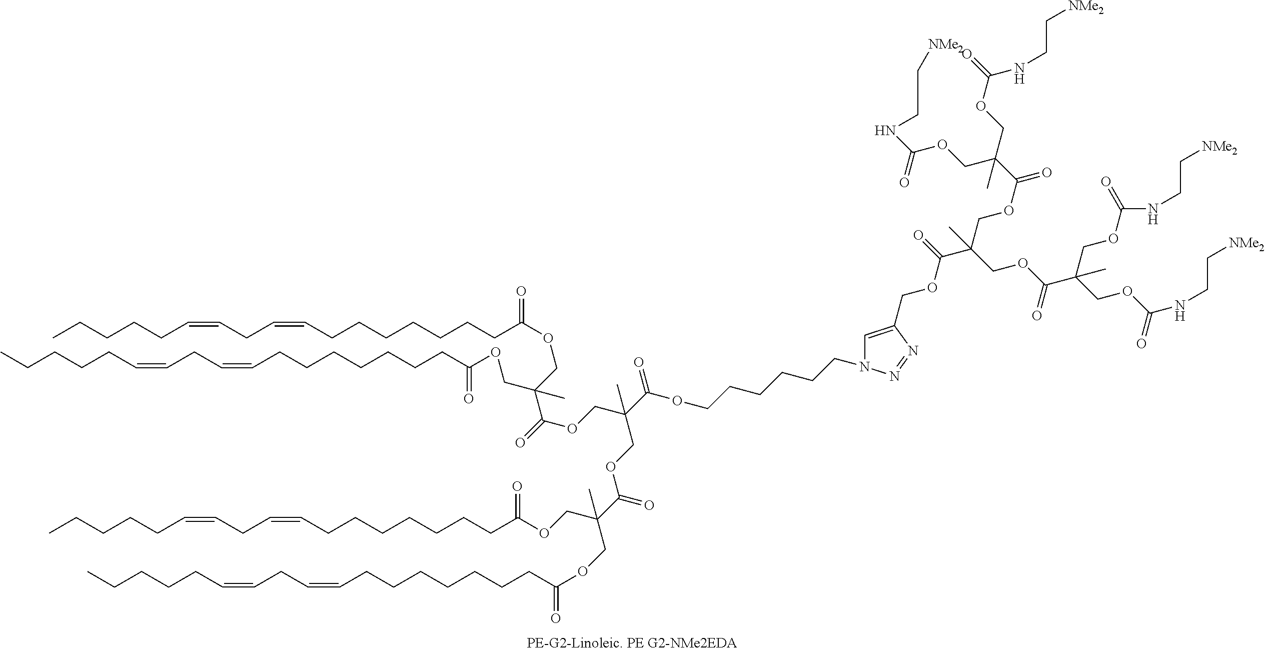 [SMILES: CCCCC/C=C\C/C=C\CCCCCCCC(=O)OCC(C)(COC(=O)CCCCCCC/C=C\C/C=C\CCCCC)C(=O)OCC(C)(COC(=O)C(C)(COC(=O)CCCCCCC/C=C\C/C=C\CCCCC)COC(=O)CCCCCCC/C=C\C/C=C\CCCCC)C(=O)OCCCCCCn1cc(COC(=O)C(C)(COC(=O)C(C)(COC(=O)NCCN(C)C)COC(=O)NCCN(C)C)COC(=O)C(C)(COC(=O)NCCN(C)C)COC(=O)NCCN(C)C)nn1]

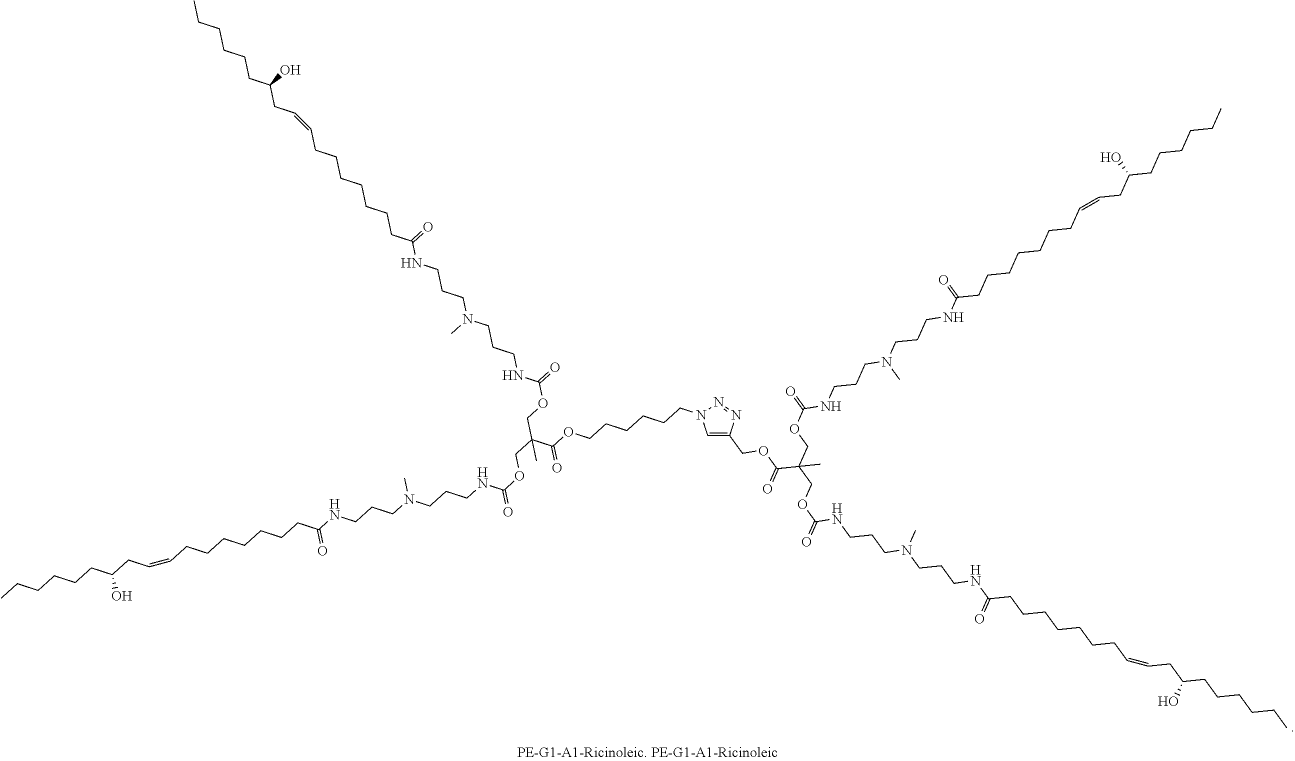 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@H](O)CCCCCC)C(=O)OCc1cn(CCCCCCOC(=O)C(C)(COC(=O)NCCCN(C)CCCNC(=O)CCCCCCC/C=C\C[C@H](O)CCCCCC)COC(=O)NCCCN(C)CCCNC(=O)CCCCCCC/C=C\C[C@H](O)CCCCCC)nn1]

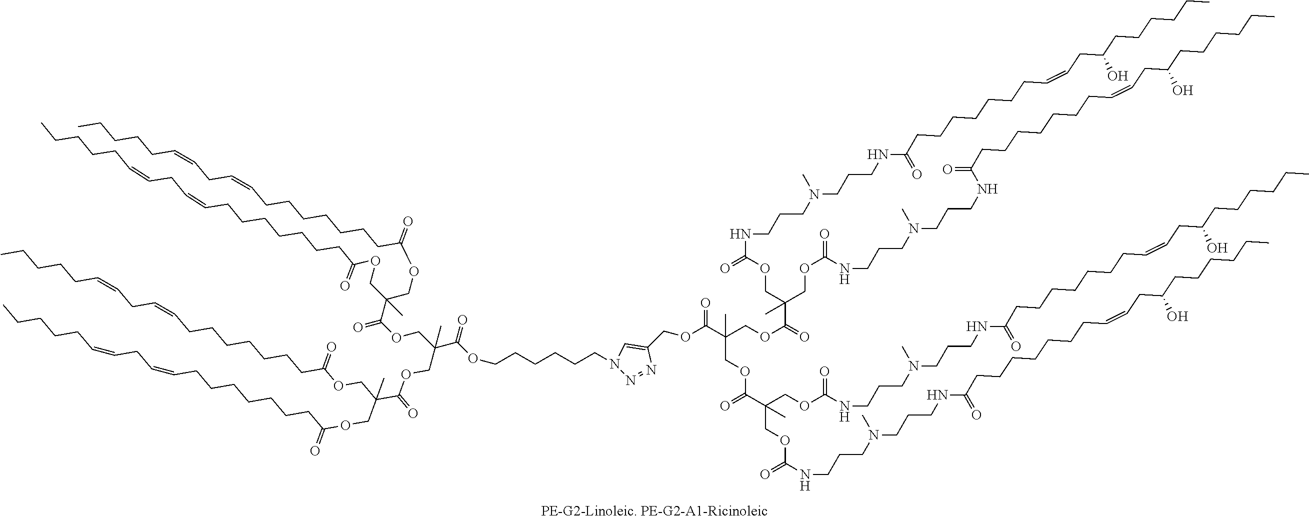 [SMILES: CCCCC/C=C\C/C=C\CCCCCCCC(=O)OCC(C)(COC(=O)CCCCCCC/C=C\C/C=C\CCCCC)C(=O)OCC(C)(COC(=O)C(C)(COC(=O)CCCCCCC/C=C\C/C=C\CCCCC)COC(=O)CCCCCCC/C=C\C/C=C\CCCCC)C(=O)OCCCCCCn1cc(COC(=O)C(C)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)nn1]

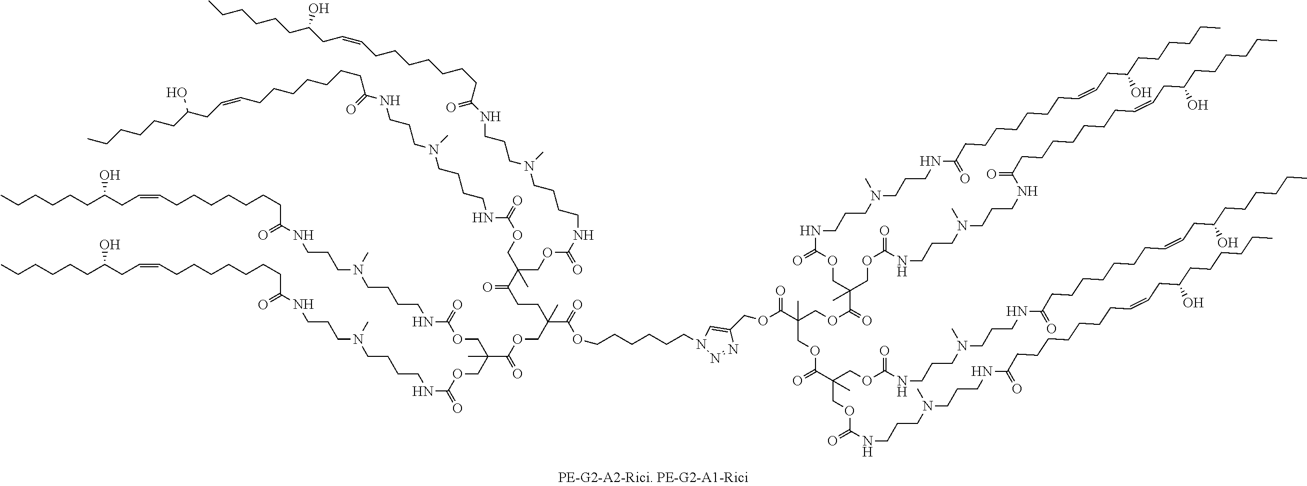

The amount of the carrier in the nanoparticle is proportional to the nucleic acid agent. The weight ratio of the carrier to the nucleic acid agent is typically in the range of 0.1:1 to 30:1 (e.g., 6:1 and 10:1). The nanoparticle preferably contains 5 wt % to 99 wt % (e.g., 10 wt % to 80 wt % and 15 wt % to 65 wt %) of the carrier as measured by the weight of the nanoparticle.

### PEG-Lipid Conjugates

The nanoparticle of this invention contains a lipid conjugate, preferably a PEG-lipid conjugate. Not to be bound by any theory, the lipid conjugate prevents the aggregation of the nanoparticles. Suitable lipid conjugates include polyethylene glycol (PEG)-lipid conjugates, such as PEG coupled to lipids (for example, DMG-PEG 2000), PEG coupled to phospholipids (for example, phosphatidylethanolamine (PEG-PE)), PEG conjugated to cholesterol or a derivative thereof, and mixtures thereof. In certain instances, the PEG may be optionally substituted by an alkyl, alkoxy, acyl, or aryl group.

Useful PEGs in the PEG-lipid conjugates are classified by their molecular weights; for example, PEG 2000 has an average molecular weight of about 2,000 Daltons, and PEG 5000 has an average molecular weight of about 5,000 Daltons. PEGs are commercially available from Avanti Polar Lipids. The PEG moiety of the PEG-lipid conjugates described herein may comprise an average molecular weight ranging from 550 Daltons to 10,000 Daltons.

Suitable lipids for conjugating with PEG include phosphatidylethanolamines having a variety of acyl chain groups of varying chain lengths and degrees of saturation. Phosphatidylethanolamines are commercially available or can be isolated or synthesized using conventional techniques. The phosphatidylethanolamines can comprise saturated or unsaturated fatty acids with carbon chain lengths in the range of C₁₀ to C₂₀. The phosphatidylethanolamines can contain mono- or polyunsaturated fatty acids and mixtures of saturated and unsaturated fatty acids. The phosphatidylethanolamines contemplated include, but are not limited to, dimyristoylphosphatidylethanolamine (DMPE), dipalmitoyl-phosphatidylethanol amine (DPPE), dioleoylphosphatidylethanolamine (DOPE), and distearoylphosphatidylethanolamine (DSPE).

The nanoparticle contains 0.5 wt % to 10 wt % (e.g., 1 wt % to 10 wt % and 1 wt % to 5 wt %) of the PEG-lipid as measured by the weight of the nanoparticle. Not to be bound by any theory, the PEG-lipid help to reduce the size of the nanoparticle and improve delivery efficiency as compared to nanoparticles lacking a PEG-lipid.

### Amphipathic Lipids

The nanoparticle also contains an amphipathic lipid. As used herein, the term “amphipathic lipid” refers to any material having non-polar hydrophobic units or “tails”, and polar “heads.” Polar groups may include, but are not limited to, phosphate, carboxylic, sulfato, amino, sulfhydryl, nitro, and hydroxyl. Nonpolar groups may include, but are not limited to, long-chain saturated and unsaturated aliphatic hydrocarbon groups and such groups substituted by one or more cycloalkyl, cycloalkenyl, aryl, heteroaryl, or heterocycle group(s). Examples of amphipathic lipids include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine. Representative examples of the phosphatidylcholine include, but are not limited to, dipalmitoylphosphatidyl choline, dioleoylphos-phatidylcholine, distearoylphosphatidylcholine, and dilinoleoylphosphatidylcholine. Representative examples of the phosphatidylethanolamine include, but are not limited to, 1,2-dioleoyl-sn-glycero-3-phosphoethanolamine or DOPE.

The nanoparticle contains the amphipathic lipid in the amount preferably ranging from 0.1 wt % to 25 wt % (e.g., 1 wt % to 25 wt %, 0.5 wt % to 20 wt %, and 1 wt % to 15 wt %) as measured by the weight of the nanoparticle. As an illustration, the amphipathic lipid constitutes 1 wt %, 3 wt %, 5 wt %, 10 wt %, 11 wt %, 12 wt %, 13 wt %, 14 wt %, or 15 wt %, or a value in a range between any two of the foregoing.

### Cholesterol or Cholesterol Derivatives

The nanoparticle further contains cholesterol or cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety and/or amino acids. In an embodiment the amino acids are selected from serine, threonine, lysine, histidine, arginine or their derivatives.

The nanoparticle includes the cholesterol or cholesterol derivative in an amount typically ranging from 1 wt % to 50 wt % (e.g., 2 wt % to 40 wt %, 3 wt % to 30 wt %, and 5 wt % to 25 wt %) of the nanoparticle. The cholesterol or cholesterol derivative wt % may be 4, 6, 8, 10, 12, 15, 20, 25, 30, 35, 40, 45, or 50 wt % or a value in a range between any two of the foregoing.

### Metal Salts

The nanoparticle contains in its core one or more metal salts in the amount ranging from 0.01 wt % to 90 wt % (e.g., 0.01 wt % to 90 wt %, 0.1 wt % to 80 wt %, 0.2 wt % to 75 wt %, 0.3 wt % to 70 wt %, 0.4 wt % to 60 wt %, 0.5 wt % to 55 wt %, and 0.1 wt % to 55 wt %) of the nanoparticle. As an illustration, the metal salt constitutes, by weight of the nanoparticle, 0.01, 0.6, 0.8, 1, 2, 5, 10, 15, 20, 25, 30, 35, 40, 45, 50, and 90 wt % or a value in a range between any two of the foregoing.

Preferred metal salts each have a divalent or trivalent metal ion such as alkaline earth metal salts, transitional metal salts, and post-transition metal salts. Examples include calcium chloride, magnesium chloride, manganese chloride, barium chloride, copper chloride, zinc chloride, calcium hydroxide, magnesium hydroxide, manganese hydroxide, barium hydroxide, copper hydroxide, zinc hydroxide, calcium sulfate, magnesium sulfate, manganese sulfate, barium sulfate, copper sulfate, zinc sulfate, aluminum chloride, aluminum sulfate, aluminum hydroxide, ferric chloride, ferric sulfate, ferric hydroxide.

Certain terminology is used in the following description for convenience only and is not limiting.

A range expressed as being between two numerical values, one as a low endpoint and the other as a high endpoint, includes the values between the numerical values and the low and high endpoints. Embodiments herein include subranges of a range herein, where the subrange includes a low and high endpoint of the subrange selected from any increment within the range selected from each single increment of the smallest significant figure, with the condition that the high endpoint of the subrange is higher than the low endpoint of the subrange.

Further embodiments herein include replacing one or more “including” or “comprising” in an embodiment with “consisting essentially of” or “consisting of.” “Including” and “comprising,” as used herein, are open ended, include the elements recited, and do not exclude the addition of one or more other element. “Consisting essentially of” means that addition of one or more element compared to what is recited is within the scope, but the addition does not materially affect the basic and novel characteristics of the combination of explicitly recited elements. “Consisting of” refers to the recited elements, but excludes any element, step, or ingredient not specified.

The words “a” and “one,” as used in the claims and in the corresponding portions of the specification, are defined as including one or more of the referenced items unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

The term “alkyl” herein refers to a straight or branched hydrocarbon group, containing 1-20 (e.g., 1-10 and 1-6) carbon atoms. Examples include methyl, ethyl, n-propyl, i-propyl, n-butyl, i-butyl, and t-butyl. Alkyl includes its halo substituted derivatives, i.e., haloalkyl, which refers to alkyl substituted with one or more halogen (chloro, fluoro, bromo, or iodo) atoms. Examples include trifluoromethyl, bromomethyl, and 4,4,4-trifluorobutyl. The term “alkoxy” refers to an —O-alkyl group. The term “acyl” refers to a —C(O)-alkyl group.

The term “cycloalkyl” refers to a saturated and partially unsaturated monocyclic, bicyclic, tricyclic, or tetracyclic hydrocarbon group having 3 to 12 carbons. Examples of cycloalkyl groups include, but are not limited to, cyclopropyl, cyclobutyl, cyclopentyl, cyclopentenyl, cyclohexyl, cyclohexenyl, cycloheptyl, and cyclooctyl.

The term “heterocycle” refers to a nonaromatic 5-8 membered monocyclic, 8-12 membered bicyclic, or 11-14 membered tricyclic ring system having one or more heteroatoms (e.g., O, N, P, and S). Examples of heterocycloalkyl groups include, but are not limited to, piperazinyl, imidazolidinyl, azepanyl, pyrrolidinyl, dihydrothiadiazolyl, dioxanyl, morpholinyl, tetrahydropuranyl, and tetrahydrofuranyl.

The term “aryl” refers to a 6-carbon monocyclic, 10-carbon bicyclic, 14-carbon tricyclic aromatic ring system wherein each ring may have 1 to 5 substituents. Examples of aryl groups include phenyl, naphthyl, and anthracenyl. The term “aralkyl” refers to alkyl substituted with an aryl group.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl. The term “heteroaralkyl” refers to an alkyl group substituted with a heteroaryl group.

The terms “halo” refers to a fluoro, chloro, bromo, or iodo radical. The term “amino” refers to a radical derived from amine, which is unsubstituted or mono-/di-substituted with alkyl, aryl, cycloalkyl, heterocycloalkyl, or heteroaryl.

The term “compound” also covers its salts and solvates. A salt can be formed between an anion and a positively charged group (e.g., amino) on a compound; examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group; examples of a suitable cation include sodium ion, potassium ion, magnesium ion, calcium ion, and an ammonium cation such as tetramethylammonium ion. Further, a salt can contain quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

In addition to the nucleic acid agent, the nanoparticle preparation of this invention also contains a pharmaceutically acceptable carrier. Preparation of such pharmaceutical compositions including a pharmaceutically acceptable carrier are known in the art. See, e.g., Remington: The Science and Practice of Pharmacy, A. Adejare, Editor, 23rd Edition, Academic Press, 2020.

The nanoparticles and compositions of this invention are useful as transfection agents. They are also useful in treating medical conditions.

As such, another aspect of this invention relates to use of the nanoparticle or the nanoparticle preparation for treating or preventing a disease, disorder or condition or for the manufacture of a medicament for treating a disease, disorder or condition.

The method for treating or preventing a disease, disorder or condition includes at least the step of administering a therapeutically effective amount of a nanoparticle or its composition of this invention to a subject in need thereof.

The term “subject” means a human or animal. Preferably, the animal is a vertebrate such as a primate, rodent, domestic animal, or game animal. Primates include chimpanzees, cynomolgus monkeys, spider monkeys, and macaques, e.g., Rhesus. The rodent may be selected from mice, rats, guinea pigs, woodchucks, ferrets, rabbits, and hamsters. The domestic or game animals may be selected from cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon. The terms, “patient” and “subject” are used interchangeably herein. A patient or subject may be selected from the foregoing or a subset of the foregoing. A patient or subject may be selected from all of the above, but excluding one or more groups or species such as humans, primates, or rodents. In an embodiment, the patient or subject may be a mammal, e.g., a primate, e.g., a human. Preferably, the subject is a mammal. The mammal may be a human, non-human primate, mouse, rat, rabbit, dog, cat, horse, pig, sheep, or cow, but is not limited to these examples. Mammals other than humans may be subjects that represent animal models of a disease or disorder. In addition, the methods described herein may be directed to treating domesticated animals and/or pets. A subject may be male or female.

As used herein, the term “therapeutically effective amount” refers to the amount of nanoparticle preparation which is effective for producing a desired therapeutic effect. The therapeutic effect may be achieved at a reasonable benefit/risk ratio applicable to medical treatment. A “therapeutically effective amount” may refer to an amount sufficient to generate appearance of antigen-specific antibodies in serum. A “therapeutically effective amount” may refer to an amount sufficient to cause a decrease in disease symptoms. A “therapeutically effective amount” may refer to an amount sufficient to cause a disappearance of disease symptoms. When treating viral infection, a decrease of disease symptoms may be assessed by decrease of virus in feces, in bodily fluids, or in secreted products. The nanoparticle preparations may be administered using an amount and by a route of administration effective for generating an immune response.

Therapeutic efficacy may depend on effective amounts of active agents and time of administration necessary to achieve a desired result. Administering a nanoparticle preparation may be a preventive measure. Administering of a nanoparticle preparation may be a therapeutic measure to promote immunity to the infectious agent, to minimize complications associated with the slow development of immunity especially in patients with a weak immune system, the elderly, or infants.

The exact dosage may be chosen by the clinician based on a variety of factors and in view of individual patients. Dosage and administration may be adjusted to provide sufficient levels of the active agent or agents or to maintain the desired effect. For example, factors which may be taken into account may include the type and severity of a disease; age and gender of the patient; drug combinations; and an individual response to therapy.

As used herein, the terms “administer,” “administering,” “administration,” or the like refer to the placement of a composition into a subject. The administration may be by a method or route which results in at least partial localization of the composition at a desired site. Placement at a desired site may lead to a production of a desired effect. A nanoparticle preparation described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” includes without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebral, and intrasternal injection and infusion. In an embodiment, the compositions may be administered by intravenous infusion or injection.

Administering a nanoparticle or its composition may be a preventive measure or a therapeutic measure, such as to promote immunity to an infectious agent, to minimize complications associated with the slow development of immunity especially in patients with a weak immune system, the elderly, or infants.

A nanoparticle or its composition may deliver a nucleic acid agent to a subject in an amount effective to vaccinate the subject from one or more diseases and disorders. The nanoparticle or its composition may serve as a vaccination platform for, without limitation, cancer or microbial pathogens, such as bacterial, viral, fungal and protozoan pathogens.

The nanoparticle or its composition may be used to immunize a subject against cancer or to treat a subject diagnosed with cancer (i.e., as a therapeutic vaccine), or to a subject having a predisposition or risk of developing cancer (i.e., as a prophylactic vaccine). Any cancer can be treated by a nanoparticle or composition of this invention using a known or new cancer therapeutic agent included in the nanoparticle or composition. Such treatment methods or their development are well-known in the art. Specific cancer treatment includes those targeting immune checkpoint or inhibitory pathways including CTLA-4 (e.g., ipilimumab for treating melanoma), Programmed Death-1 (PD-1), CD28, ICOS, BTLA, Programmed Death Ligand-1 (PD-L1), Programmed Death Ligand-2 (PD-L2), PD-1/PD-L1, lymphocyte-activation gene 3 (LAG3), TIM3, VISTA, OX40, 4-1BB.

Cancer examples include melanoma (MEL), squamous non-small cell lung cancer (NSCLC), non-squamous NSCLC, triple-negative breast cancer (TNBC), renal cell carcinoma (RCC), colorectal cancer (CRC), castration-resistant prostate cancer (CRPC), hepatocellular carcinoma (HCC), squamous cell carcinoma of the head and neck, carcinomas of the esophagus, ovary, gastrointestinal tract and breast, or a hematologic malignancy such as multiple myeloma, B-cell lymphoma, T-cell lymphoma, Hodgkin's lymphoma/primary mediastinal B-cell lymphoma, and chronic myelogenous leukemia.

The nanoparticle or its composition can be administered to a cancer patient in addition to one or more additional therapeutic agents. In some embodiments, bioinformatics may be used to sequence each patient's unique tumor exome to identify neoantigens. Then, corresponding mRNAs of these neoantigens may be used to generate the antigens necessary to create immunity. Finally, these mRNAs may be delivered via a nanoparticle preparation.

The nanoparticle or its composition may include a nucleic acid agent encoding one or more tumor antigens. It may be used to provide immunity and therapeutic activity against tumor cells and non-tumor cells located within a tumor or a tumor environment. Further, it may provide protective and/or therapeutic activity against solid tumors and cancers of the blood. Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or ureter); Wilms' tumor; bladder cancers including, but not limited to, transitional cell carcinoma, squamous cell cancer, adenocarcinoma, carcinosarcoma.

The nanoparticle or its composition may deliver a nucleic acid agent encoding antigens to a subject in an amount effective to vaccinate the subject from one or more infectious diseases caused by a wide variety of microbial pathogens, such as bacterial, viral, fungal and protozoan pathogens. In some embodiments, the target of a vaccine could be any of a large number of microbial pathogens. Exemplary diseases that can be vaccinated against include disease for which vaccines are currently available, including Anthrax; Diseases (e.g., cervical cancer, cancer of the esophagus) caused by Human Papillomavirus (HPV); Diphtheria; Hepatitis A; Hepatitis B; Haemophilus influenzae type b (Hib); Influenza viruses (Flu); Japanese encephalitis (JE); Lyme disease; Measles; Meningococcal; Monkeypox; Mumps; Pertussis; Pneumococcal; Polio; Rabies; Rotavirus; Rubella; Shingles (Herpes Zoster); Smallpox; Tetanus; Toxoplasmosis; Typhoid; Tuberculosis (TB); Varicella (Chickenpox); Yellow Fever. In some embodiments, nanoparticle preparations may be used to immunize a subject against an infectious disease or pathogen for which no alternative vaccine is available, such as diseases including, but not limited to, malaria, streptococcus, Ebola Zaire, HIV, Herpes virus, hepatitis C, Middle East Respiratory Syndrome (MERS), Sleeping sickness, Severe Acute Respiratory Syndrome (SARS), coronavirus disease 2019 (COVID-19), rhinovirus, chicken pox, Hendra virus, Nipah virus, Zika virus, respiratory syncytial virus (RSV), influenza virus, Mycobacterium tuberculosis, parasites of the genus Plasmodium (e.g., malaria), and others.

The nanoparticle or its composition may deliver a therapeutic agent effective against an allergic condition including food allergies, asthma, and atopic dermatitis. Further, it can deliver a therapeutic agent useful in treating heart diseases or other cardiovascular conditions including coronary artery disease (CAD), hypertension (high blood pressure), heart failure (congestive heart failure), myocardial infarction (heart attack), and arrhythmias such as atrial fibrillation (AFib), ventricular tachycardia, bradycardia, cardiomyopathy, congenital heart disease, valvular heart disease, peripheral artery disease (PAD), aortic aneurysm, pericarditis, endocarditis, sudden cardiac arrest, pulmonary hypertension, rheumatic heart disease, atrial septal defect (ASD), ventricular septal defect (VSD), Takotsubo cardiomyopathy, mitral valve prolapse, cardiac/cardiovascular diseases such as Danon disease, coronary artery disease (CAD), hypertension, congestive heart failure, myocardial infarction, arrhythmias, cardiac inflammation, and dilated cardiomyopathy. The delivered therapeutic agent may be otherwise improve cardiac function associated with these heart diseases.

The above-mentioned examples are provided illustratively, as this invention has a broad disease application and is not limited to the above examples.

The nanoparticle or its composition may be administered alone, or in combination with one or more additional active agents, as part of a therapeutic or prophylactic treatment regime. A nanoparticle preparation may be administered on the same day, or a different day than a second active agent. As used herein, “combination” or “combined” means either concomitant, simultaneous, or sequential administration of two or more agents. In some embodiments, combinations may be administered concomitantly (e.g., as a mixture), separately but simultaneously (e.g., via separate intravenous lines into the same subject), or sequentially (e.g., one compound or agent is given first followed by a second compound or agent). In some embodiments, an additional prophylactic or therapeutic agent may be a vaccine for a specific antigen, which may be the same or different than an antigen encoded by a nucleic acid agent.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever.

All publications, including patent documents, cited herein are incorporated by reference in their entirety.

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Example 1. Calcium or Magnesium Ion Incorporation into Dendrimer-1 Nanoparticles Enhanced the Expression of SEAP in Baby Hamster Kidney (BHK) Cells

Nanoparticle Preparation with or without Magnesium or Calcium Salt:

Nanoparticles of this invention were prepared using Dendrimer-1: cholesterol, DSPC, and DMG-PEG2k at molar ratios of 1:2.88:0.6:0.065 on a NanoAssemblr® Benchtop™ system (Precision NanoSystems Inc, Vancouver, BC, Canada). RNA encoding secreted alkaline phosphatase (SEAP) was diluted with DNase/RNase-Free, endotoxin free distilled water and sterile citrate buffer. For calcium salt-containing formulations, varied amounts of calcium chloride solution were added to the RNA phase. For magnesium salt-containing formulations, MgCl₂ was added to the formulation at a mass ratio of 1.1:1 (MgCl₂ to RNA). Cholesterol and MgCl₂ were obtained from Sigma-Aldrich (St Louis, Missouri). DSPC and DMG-PEG2k were purchased from the Avanti Polar Lipids (Alabaster, Alabama). CaCl₂) was ordered from PromoCell (Heidelberg, Germany). RNA was synthesized at Tiba Biotech (Cambridge, Massachusetts). A total flow rate was maintained at 8 mL per min and a 3:1 ratio of aqueous to organic phase for formulation using the NanoAssemblr™ instrument. In glassware depyrogenated by heating at 250° C. for 24 hours, nanoparticles were dialyzed against sterile, endotoxin-free PBS using 10,000 molecular weight cutoff dialysis cassettes. Dialyzed nanoparticles were sterile filtered using 0.2 micron poly(ether sulfone) filters and characterized with a Zetasizer® NanoZS instrument (Malvern Panalytical, United Kingdom). The size distributions were characterized by a single peak with a low polydispersity index. Encapsulation efficiency was measured for the nanoparticle preparation containing Dendrimer-1 (with or without calcium or magnesium ions) and SEAP repRNA (formulated at pH 5) using Ribogreen® assay (Geall et al. 10.1073/pnas. 1209367109 which is incorporated herein by reference as if fully set forth). The encapsulation efficiency for nanoparticles based on Dendrimer-1 without calcium chloride or magnesium chloride is 100%. The encapsulation efficiency for nanoparticles based on Dendrimer-1 with calcium chloride at weight ratio to RNA payload of 1.3 and 3.3 is 100% and 94%, respectively. The encapsulation efficiency for nanoparticles based on Dendrimer-1 with magnesium chloride at weight ratio to RNA payload of 1.1 is 98%

### Hydrodynamic Size Measurement

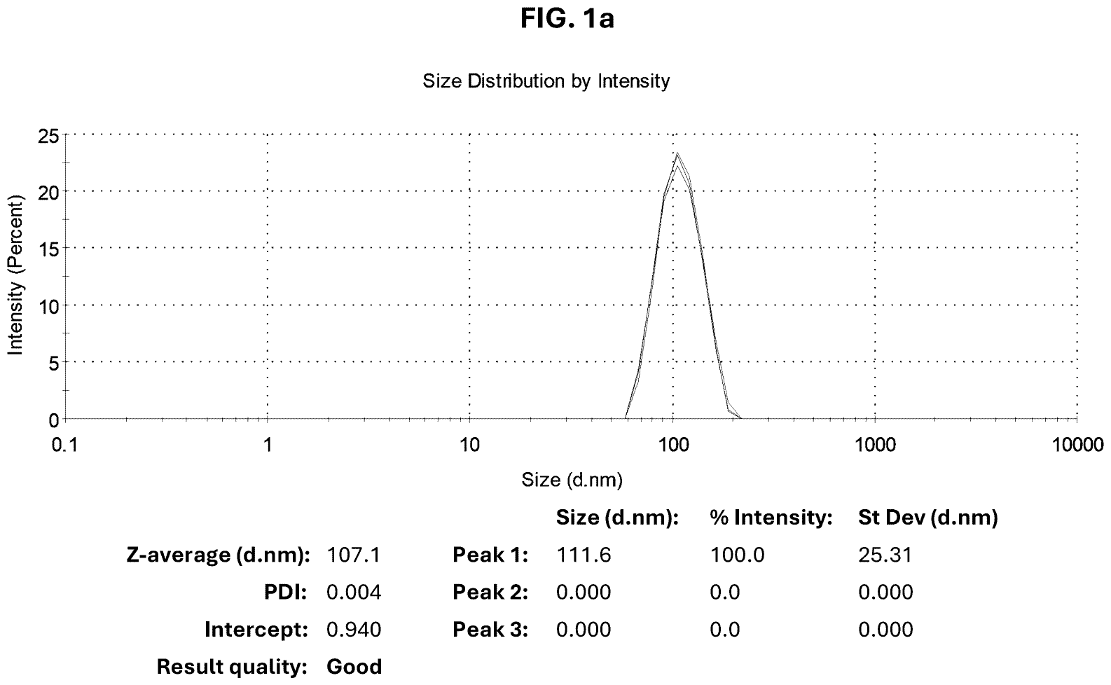

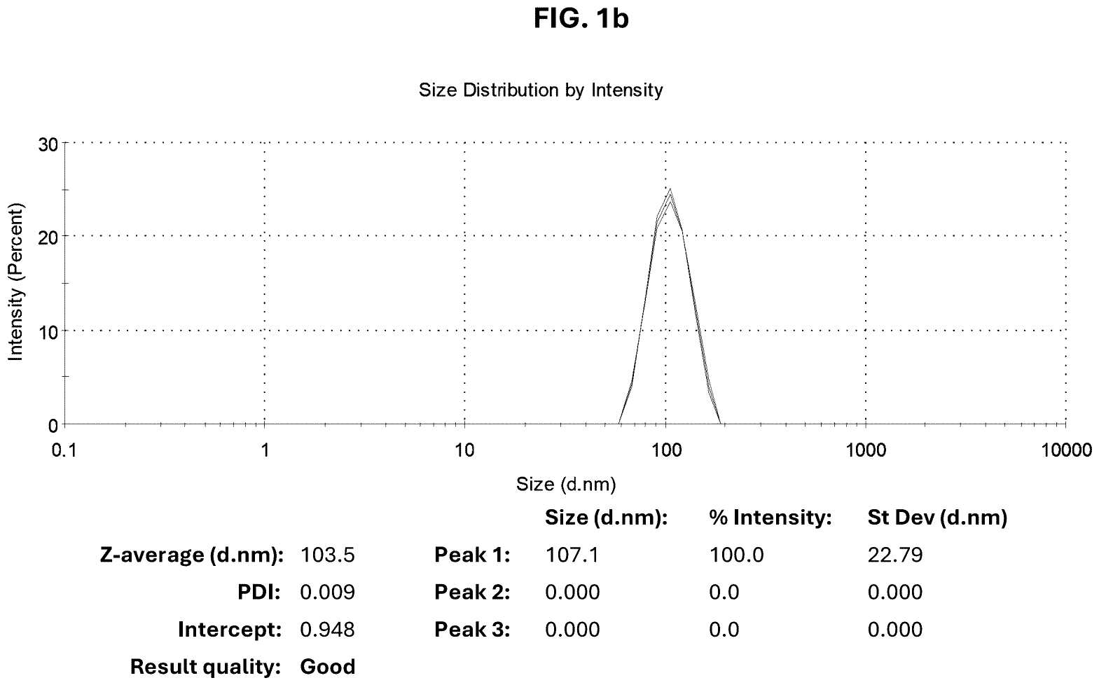

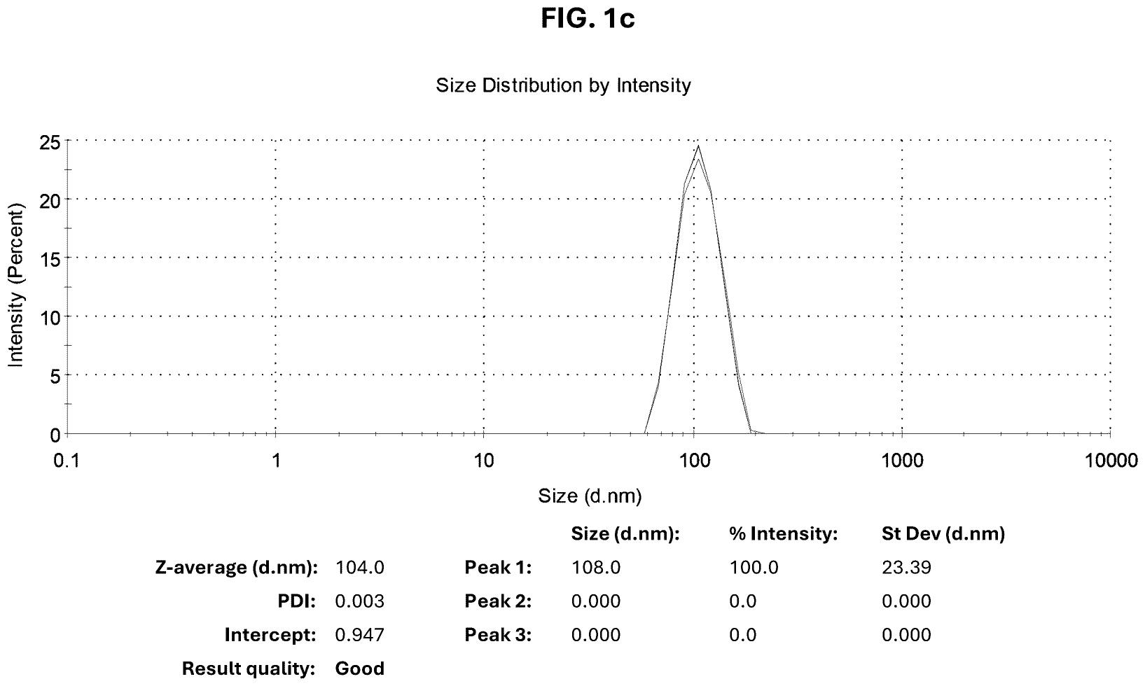

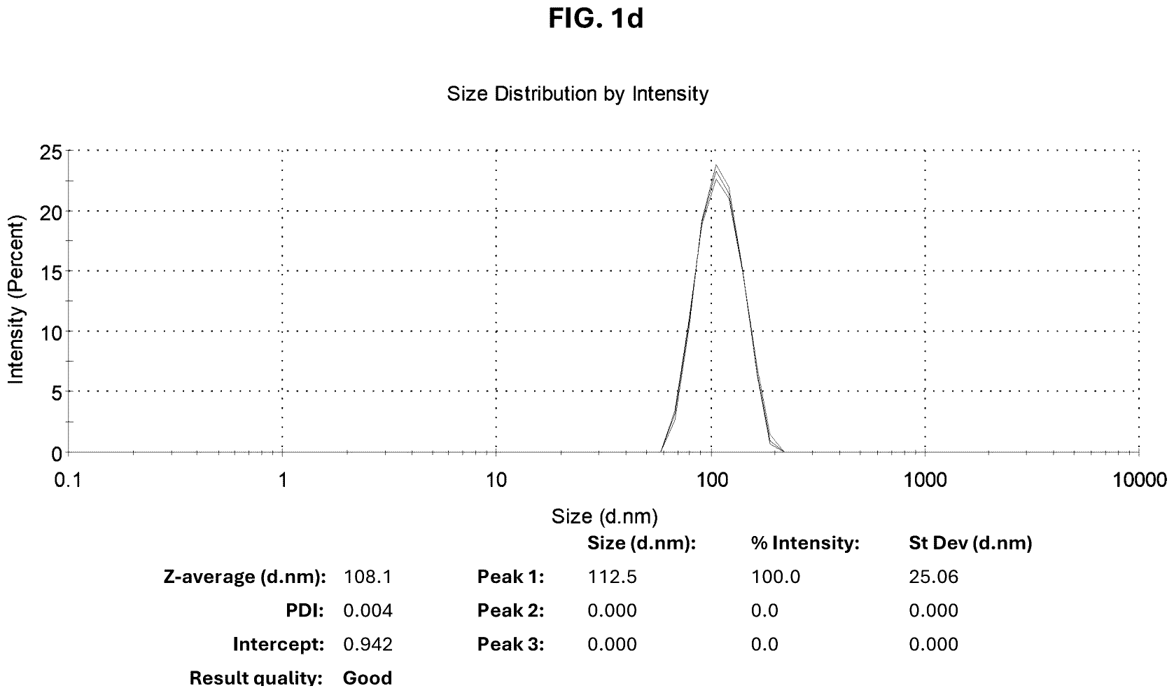

The “Z average” of the nanoparticle preparation containing Dendrimer-1, cholesterol, DSPC, DMG-PEG 2000 and SEAP repRNA, without or with different CaCl₂) or MgCl2 concentrations as function of size was determined by dynamic light scattering (DLS). Referring to FIG. 1a, the Z average for the nanoparticles without calcium salts/ions was 107.1 d.nm in size, with the buffer pH in the formulation at 5. Referring to FIG. 1b, the Z average for the nanoparticles with calcium chloride to RNA weight ratio at 1.3 was 103.5 d.nm in size, with the buffer pH in the formulation at 5. Referring to FIG. 1c, the Z average for the nanoparticles with magnesium chloride to RNA weight ratio at 1.1 was 104.0 d.nm in size, with the buffer pH in the formulation at 5. Referring to FIG. 1d, the Z average was observed for the nanoparticles with calcium chloride to RNA weight ratio at 3.3 was 108.1 d.nm in size, with the buffer pH in the formulation at 5. Referring to FIG. 1a-d, the size distributions were characterized with a low polydispersity index (<0.01), indicating monodispersity.

### In Vitro Expression of Secreted Alkaline Phosphatase (SEAP):

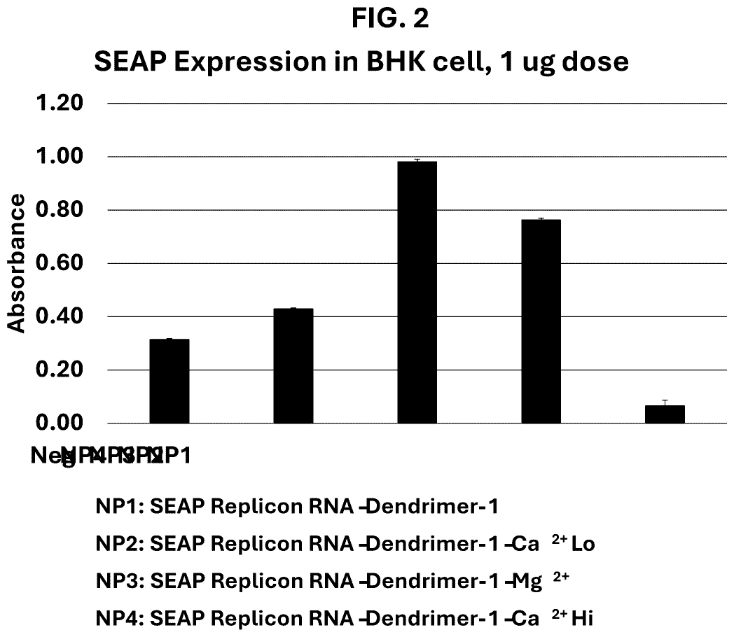

FIG. 2 shows optical density measurements showing the expression of secreted alkaline phosphatase (SEAP) with nanoparticle formulations formulated with or without ions. To test the ability of the nanoparticles formulated with Dendrimer-1 as the carrier to express SEAP in vitro, BHK cells were treated with various nanoparticles of this invention. Each well of a 12 well dish of BHKs was treated with 20 μL (approximately 1 μg) of each nanoparticle preparation diluted into a final volume of 600 μL with a 1:1 OptiMeM:PBS mixture. After the treatment, BHK cells were incubated at 37° C. and 5% CO₂. After 12 hours, the cell culture medium was collected and assayed for SEAP using the InvivoGen® QUANTI-Blue™ colorimetric assay (San Diego, CA, USA), according to the manufacturer's protocol. Briefly, 50 μL of the cell culture medium was added to 150 μL of the QUANTI-Blue™ solution and incubated at 37° C. The optical absorbance was measured at 620-655 nm using a microplate reader. FIG. 2 illustrates the SEAP expression of nanoparticle preparations containing Dendrimer-1 with different metal salts based on optical density compared to the negative control. As seen in FIG. 2, the absorbance of the nanoparticle preparation containing MgCl₂ is the highest among the tested compositions. Calcium ions, even included at a low amount, significantly increased the SEAP expression. As the calcium ion concentration was increased to a high level, the expression of the SEAP protein also increased. The data suggest that calcium and magnesium ions in the compositions enhance the expression of the protein significantly without compromising the encapsulation efficiency and size distribution of the nanoparticles.

### Example 2. Calcium Ion Incorporation into Dendrimer-1, Dendrimer-2, or Dendrimer-3 Based Nanoparticles Enhances Expression and Immunogenicity in Balb/c Mice

Nanoparticle Preparation with or without Calcium Salts:

Nanoparticles of this invention were prepared using Dendrimer-2 or Dendrimer-1 or Dendrimer-3, cholesterol, DSPC, and DMG-PEG2k at a molar ratio of 1:2.88:0.6:0.065 on a NanoAssemblr® Benchtop™ system (Precision NanoSystems Inc, Vancouver, BC, Canada). SEAP repRNA and PR8 HA repRNA at 1:1 mass ratio were diluted with DNase/RNase-Free, endotoxin free distilled water and a sterile citrate buffer to a final desired pH. For calcium salt-containing compositions, a predetermined amount of calcium chloride solution was added to the RNA phase at a mass ratio of 1.3 to the RNA. A total flow rate was maintained at 8 mL per min, and a 3:1 ratio of aqueous to organic phase volumes was used. In glassware depyrogenated by heating at 250° C. for 24 hours, the nanoparticles thus prepared were dialyzed against sterile, endotoxin-free PBS using 10,000 molecular weight cutoff dialysis cassettes. The dialyzed nanoparticles were sterile filtered using 0.2 micron poly(ether sulfone) filters and characterized with a Zetasizer® NanoZS instrument (Malvern). The size distributions were characterized by a single peak with low polydispersity indexes. Encapsulation efficiency was measured for the nanoparticle preparation containing Dendrimer-2, or Dendrimer-1 or Dendrimer-3 (with or without a calcium salt) and SEAP repRNA/PR8 HA repRNA combinations (formulated at pH 5) using Ribogreen® assay (Geall et al. 10.1073/pnas. 1209367109 which is incorporated herein by reference as if fully set forth). The encapsulation efficiency for nanoparticles based on Dendrimer-2, or Dendrimer-1 or Dendrimer-3 without calcium chloride is 99.1%, 99.0% and 99.0% respectively. The encapsulation efficiency for nanoparticles based on Dendrimer-2, or Dendrimer-1 or Dendrimer-3 with calcium chloride is 99.0%, 99.0% and 99.1% respectively.

### Hydrodynamic Size Measurement

The “Z average” of the nanoparticle preparations containing Dendrimer-2 or Dendrimer-1 or Dendrimer-3, cholesterol, DSPC, DMG-PEG 2000 and SEAP Replicon RNA/PR8 HA Replicon RNA, without or with CaCl₂):RNA weight ratio around 1.3 as function of size was determined by dynamic light scattering (DLS). The buffer pH value was at 5.

The results are shown in Table 1 below.

TABLE 1
DLS results

| Nanoparticle | Z-Ave (d · nm) | PDI | z-avg stdev | PDI-stdev |
|---|---|---|---|---|
| NP1 | 107.5 | 0.044 | 0.5 | 0.02 |
| NP2 | 107.0 | 0.029 | 0.2 | 0.01 |
| NP3 | 103.2 | 0.034 | 0.2 | 0.01 |
| NP4 | 102.6 | 0.023 | 0.5 | 0.02 |
| NP5 | 107.4 | 0.024 | 1.0 | 0.02 |
| NP6 | 108.5 | 0.011 | 0.5 | 0.01 |

As shown in the table above, NP1, the Z average for the Dendrimer-2 nanoparticles without a calcium salt was 107.5 d.nm in size; NP3, the Z average for the Dendrimer-1 nanoparticles without a calcium salt was 103.2 d.nm in size; and NP5, the Z average for the Dendrimer-3 nanoparticles without a calcium salt was 107.4 d.nm in size. By contrast, NP2, the Z average for the Dendrimer-2 nanoparticles with calcium chloride to RNA weight ratio at ˜1.3 was 107 d.nm in size; NP4, the Z average for the Dendrimer-1 nanoparticles with calcium chloride to RNA weight ratio at ˜1.3 was 102.6 d.nm in size; and NP6, the Z average for the Dendrimer-2 nanoparticles with calcium chloride to RNA weight ratio at ˜1.3 was 108.5 d.nm in size. The size distributions in all compositions were characterized by a low polydispersity index (<0.05), indicating monodispersity.

### In Vivo Study: Calcium Ions Enhance the Expression of SEAP

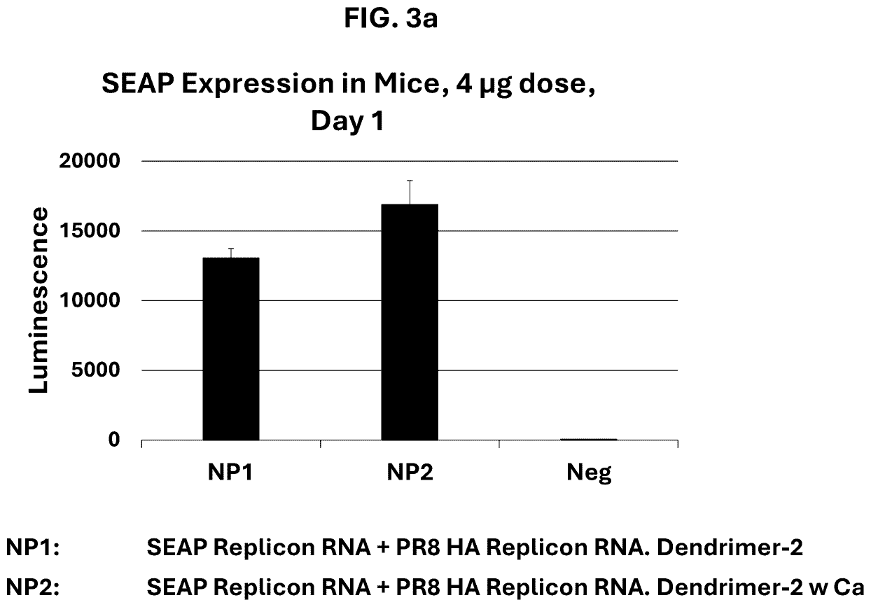

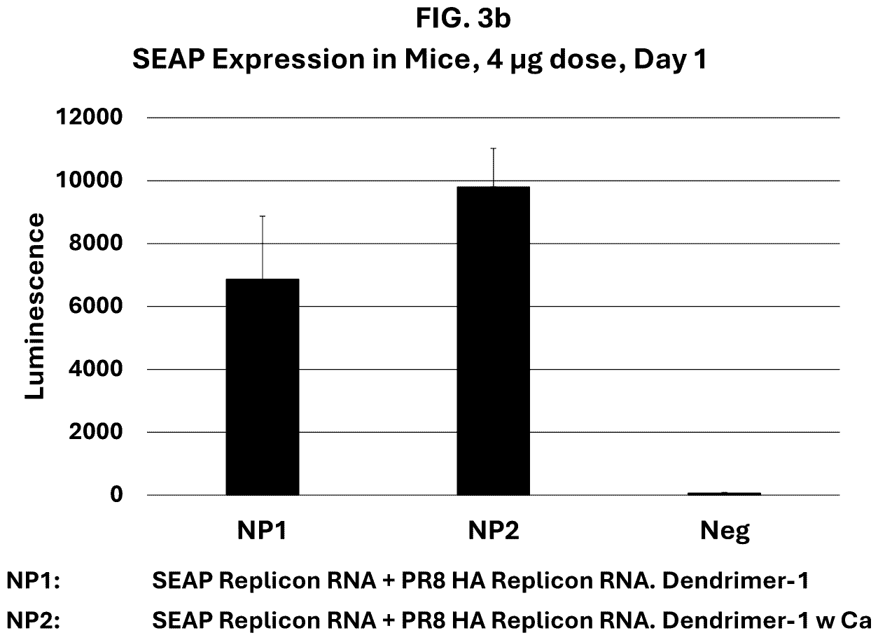

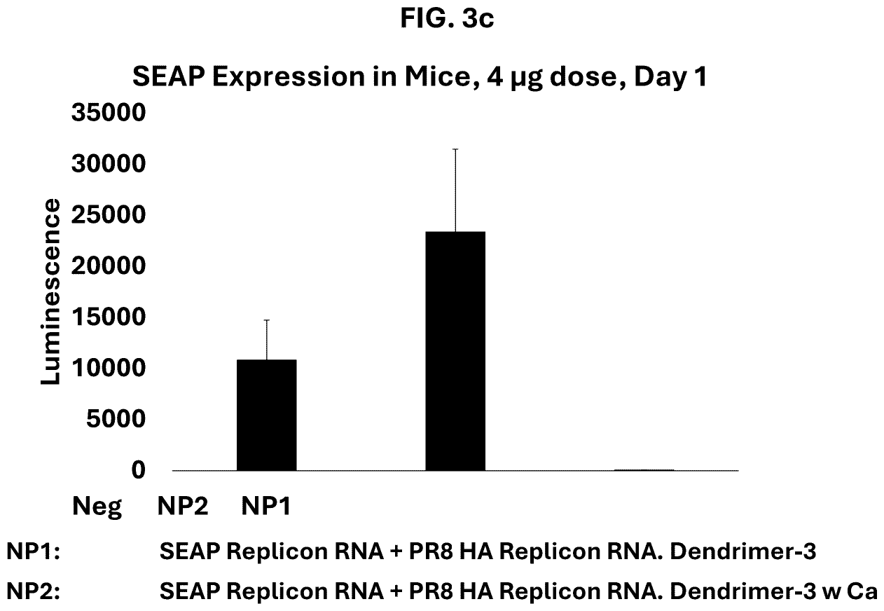

Mice were injected with 4 μg (2 μg of SEAP repRNA and 2 μg of PR8 HA repRNA) of each nanoparticle preparation at an RNA concentration of 0.04 μg/μL. After treatment (day 1), Phospha-Light™ assay was used to quantify SEAP protein level in serum. As seen in FIG. 3a, the SEAP expression induced by Dendrimer-2 nanoparticles containing calcium ions (formulated at pH 5) is higher than that induced by Dendrimer-2 nanoparticles without calcium ions. Now referring to FIG. 3b, the SEAP expression induced by Dendrimer-1 nanoparticles containing calcium ions (formulated at pH 5) is higher than that induced by Dendrimer-1 nanoparticles without calcium ions. Now referring to FIG. 3c, the SEAP expression induced by Dendrimer-3 nanoparticles with calcium ions (formulated at pH 5) is higher than that induced by Dendrimer-3 nanoparticles without calcium ions.

### Calcium Ion Incorporation Enhances Immunogenicity

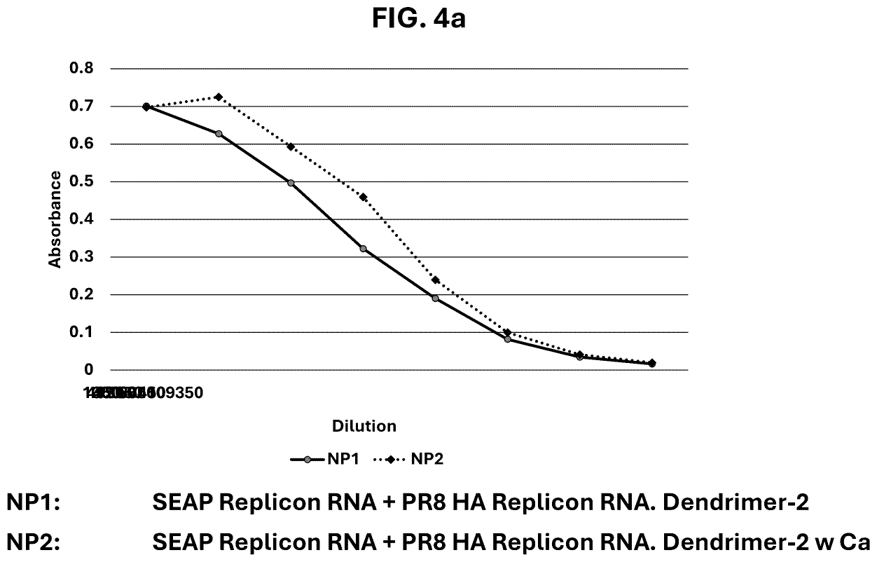

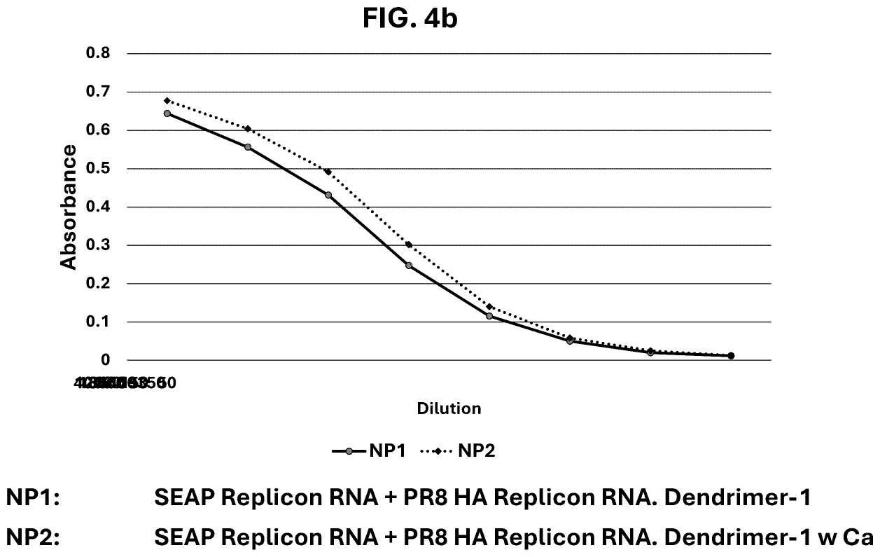

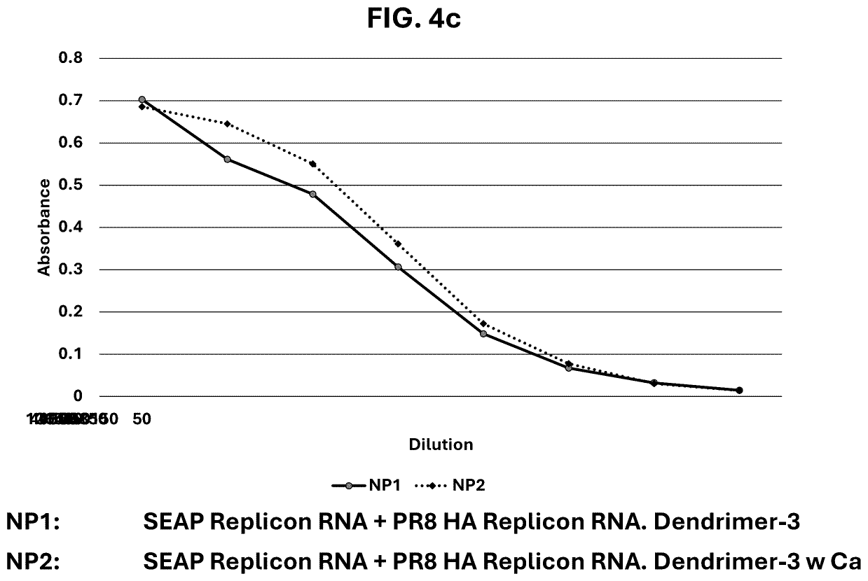

Briefly, animals were injected with 4 μg (2 μg of SEAP repRNA and 2 μg of PR8 HA repRNA) of each nanoparticle preparation at a concentration of 0.04 μg/μL. On day 14 post-treatment (week 2), the serum was collected and diluted for anti-HA IgG ELISA assay. As seen in FIG. 4a, serum from animals treated with the nanoparticle preparation containing Dendrimer-2 and calcium ions exhibited higher absorbance across dilutions than that of the nanoparticle preparation containing Dendrimer-2 but not calcium ions. As seen in FIG. 4b, the nanoparticle preparation containing Dendrimer-1 and calcium ions showed higher absorbances than that of the nanoparticle preparation containing Dendrimer-1 but not calcium ions. As seen in FIG. 4c, the nanoparticle preparation containing Dendrimer-3 and calcium ions showed higher absorbances than that of the nanoparticle preparation containing Dendrimer-3 but not calcium ions. The higher absorbance indicate higher serum IgG responses specific to PR8 HA protein. The results demonstrate that metal ions improve immunogenicity of the nanoparticles and thus superior performance as a vaccine.

### Example 3. Calcium Ion Incorporation into Dendrimer-4 Based Nanoparticles Leads to a Highly Specific Expression of Protein in Heart

Nanoparticles Prepared with or without Calcium Salts:

Nanoparticles of this invention were prepared using Dendrimer-4, cholesterol, DOPE, and DMG-PEG2k at a molar ratio of 1:2.88:0.6:0.075 on a NanoAssemblr® Benchtop™ system (Precision NanoSystems Inc, Vancouver, BC, Canada). Luciferase repRNA was diluted with DNase/RNase-Free, endotoxin-free distilled water and sterile citrate buffer. For calcium salt-containing compositions, a predetermined amount of calcium chloride solution was added to the RNA phase at a mass ratio of 1.3 to the RNA. A total flow rate was maintained at 8 mL per min, and a 3:1 volume ratio of aqueous to organic phase was used. In glassware depyrogenated by heating at 250° C. for 24 hours, the nanoparticles thus prepared were dialyzed against sterile, endotoxin-free PBS using 10,000 molecular weight cutoff dialysis cassettes. The dialyzed nanoparticles were sterile filtered using 0.2 micron poly(ether sulfone) filters and characterized with a Zetasizer® NanoZS instrument (Malvern). The size distributions were characterized by a single peak with a low polydispersity index. Encapsulation efficiency was measured for the nanoparticle preparation containing Dendrimer-4 (with or without a calcium salt) and Luciferase repRNA (formulated at pH 6) using Ribogreen® assay (Geall et al. 10.1073/pnas. 1209367109 which is incorporated herein by reference as if fully set forth).

### Hydronamic Size Measurement

The “Z average” diameter of the nanoparticle preparations containing Dendrimer-4, cholesterol, DOPE, DMG-PEG 2000 and Luciferase repRNA, without or with CaCl₂):RNA weight ratio around 1.3 was determined by dynamic light scattering (DLS). The buffer pH value at formulation was 6 for these samples.

The results are shown in Table 2 below.

TABLE 2
DLS results

| Nanoparticle | Z-Ave (d · nm) | PDI | z-avg stdev | PDI-stdev |
|---|---|---|---|---|
| NP1 | 100.5 | 0.032 | 0.9 | 0.03 |
| NP2 | 100.3 | 0.080 | 1.1 | 0.03 |

As shown in Table 2 above, the Z average for the Dendrimer-4 nanoparticles without a calcium salt (NP1) was 100.5 d.nm in size and the Z average for the Dendrimer-4 nanoparticles with calcium chloride to RNA weight ratio of ˜1.3 (NP2) was 100.3 d.nm in diameter. The size distributions in all compositions were characterized with a low polydispersity index (<0.1), indicating monodispersity.

### In Vivo Study: Incorporation of Calcium Ions Changes the Biodistribution of Intravenously Administered Nanoparticles

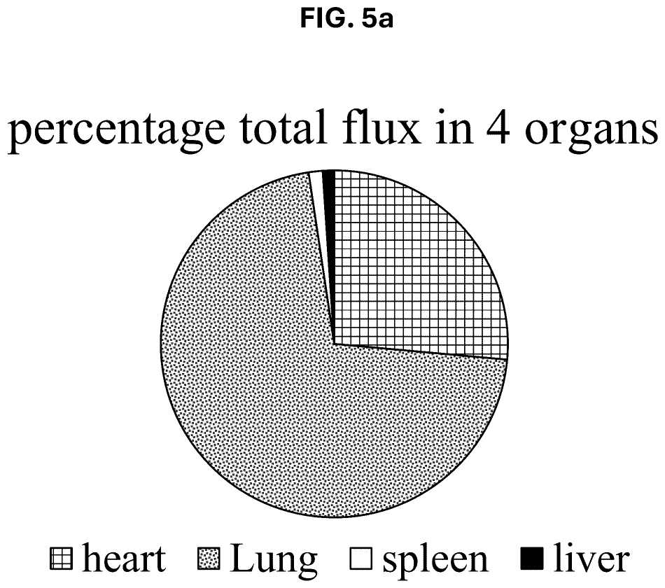

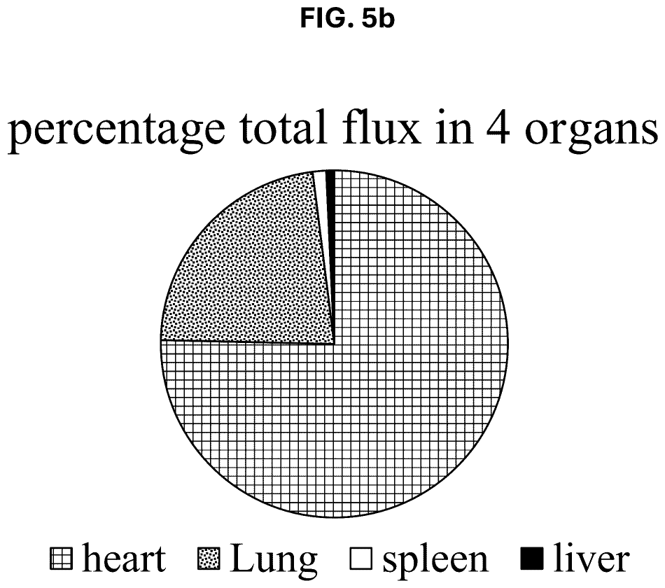

Mice were injected with 5 μg of nanoparticle-formulated Luciferase repRNA, at an RNA concentration of 0.025 μg/μL. After treatment (day 1), D-Luciferin (Syd Labs) was administered as a substrate for the expressed luciferase gene encoded by the RNA. The resulting luminescence from excised tissues was imaged with an IVIS system (PerkinElmer). As seen in FIG. 5a, the majority of luminescent signal (photon flux) induced by Dendrimer-4 without calcium ions (formulated at pH 6) is in lung (71%), with a minority portion in heart (26%). Referring the FIG. 5b, the majority (more than 75%) of photons induced by Dendrimer-4 nanoparticles containing calcium ions (formulated at pH 6) are exhibited in the heart.

### Example 4

### Nanoparticles Prepared by Adding Calcium Salts Post the Formulation:

Nanoparticles of this invention were prepared using Dendrimer-4, cholesterol, DOPE, and DMG-PEG2k at a molar ratio of 1:2.88:0.6:0.075 on a NanoAssemblr® Benchtop™ system (Precision NanoSystems Inc, Vancouver, BC, Canada). Luciferase repRNA was diluted with DNase/RNase-Free, endotoxin-free distilled water and sterile citrate buffer. A total flow rate was maintained at 8 mL per min, and a 3:1 volume ratio of aqueous to organic phase was used. In glassware depyrogenated by heating at 250° C. for 24 hours, the nanoparticles thus prepared were dialyzed against sterile, endotoxin-free PBS using 10,000 molecular weight cutoff dialysis cassettes. The dialyzed nanoparticles were sterile filtered using 0.2 micron poly(ether sulfone) filters and characterized with a Zetasizer® NanoZS instrument (Malvern). CaCl₂) was added into the dialyzed nanoparticle suspension (at theoretical wt % of 13%) and characterized with a Zetasizer® NanoZS instrument (Malvern). The size distributions for nanoparticle suspension without or with CaCl₂) were characterized by a single peak with a low polydispersity index.

### Hydronamic Size Measurement

The “Z average” diameter of the nanoparticle preparations containing Dendrimer-4, cholesterol, DOPE, DMG-PEG 2000 and Luciferase repRNA, without or with CaCl₂) (CaCl₂):RNA weight ratio around 2) was determined by dynamic light scattering (DLS).

The results are shown in Table 3 below.

TABLE 3
DLS results

| Nanoparticle | Z-Ave (d · nm) | PDI | z-avg stdev | PDI-stdev |
|---|---|---|---|---|
| NP1 | 113.7 | 0.036 | 1.0 | 0.03 |
| NP2 | 116.9 | 0.047 | 1.0 | 0.02 |

As shown in Table 3 above, the Z average for the Dendrimer-4 nanoparticles without a calcium salt (NP1) was 113.7 d.nm in size and the Z average for the Dendrimer-4 nanoparticles with calcium chloride (NP2) was 116.9 d.nm in diameter. The size distributions in all compositions were characterized with a low polydispersity index (<0.1), indicating monodispersity with narrow particle size distribution.

CryoEM Analysis of Nanoparticles with the CaCl₂) Added Post the Formulation

The morphology and internal structure of the nanoparticle NP2 were determined by Cryo-electron microscopy (CryoEM).

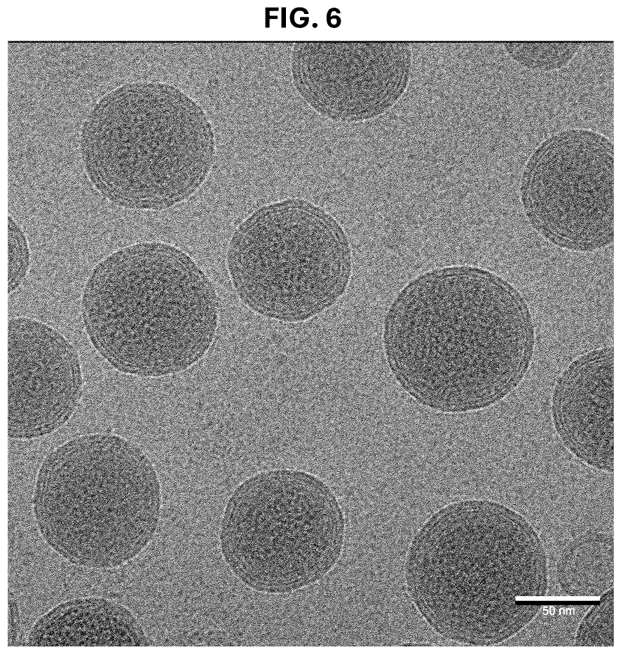

As seen in FIG. 6, the morphology of NP2 looked spherical and multilamellar. The internal structure of the nanoparticles is uniform; no bleb structure was seen. The scale bar is 50 nm.

### In Vivo Study: Incorporation of Calcium Ions Post the Formulation Changes the Biodistribution of Intravenously Administered Nanoparticles

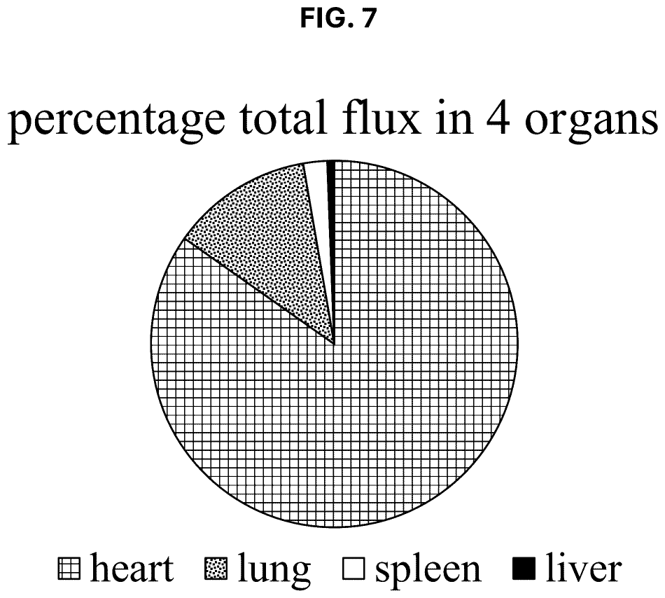

Mice were injected with 5 μg of NP2 containing Luciferase repRNA, at an RNA concentration of 0.025 μg/μL. After treatment (day 1), D-Luciferin (Syd Labs) was administered as a substrate for the expressed luciferase gene encoded by the RNA. The resulting luminescence from excised tissues was imaged with an IVIS system (PerkinElmer). Referring the FIG. 7, the majority (˜ 85%) of photons induced by Dendrimer-4 nanoparticles (formulated at pH 6) containing calcium ions were exhibited in the heart.

### OTHER EMBODIMENTS

All of the features disclosed in this specification may be combined in any combination. Each feature disclosed herein may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions.

What is claimed is:

1. A nanoparticle preparation comprising a nucleic acid agent, a nucleic acid carrier, a metal salt, a polyethylene glycol (PEG)-lipid, a phospholipid, and a cholesterol or its derivative.

2. The nanoparticle preparation of claim 1, wherein the metal salt contains a divalent or trivalent metal ion.

3. The nanoparticle preparation of claim 1, wherein the nucleic acid agent is a polynucleotide, oligonucleotide, a deoxyribonucleic acid (DNA), a complementary DNA (cDNA), a ribonucleic acid (RNA), a replicon RNA (repRNA), a small interfering RNA (siRNA), a microRNA (miRNA), a single strand guide RNA (sgRNA), a messenger RNA (mRNA), or any combination thereof.

4. The nanoparticle preparation of claim 1, wherein the nucleic acid agent encodes a protein, a polypeptide, or an oligopeptide.

5. The nanoparticle preparation of claim 4, wherein the protein is a therapeutic protein or an immunogenic protein.

6. The nanoparticle preparation of claim 5, wherein the immunogenic protein comprises one or more antigens associated with an infectious disease agent, a pathogen, or a cancer.

7. The nanoparticle preparation of claim 5, wherein the therapeutic protein is effective against an infectious disease, a pathogen, a cancer, an autoimmune disease, a cardiovascular disease, or an allergic disease.

8. The nanoparticle preparation of claim 3, wherein the nucleic acid agent is an RNA or DNA molecule capable of silencing, inhibiting, enhancing, or otherwise modifying the activity of a gene.

9. The nanoparticle preparation of claim 1, wherein the nucleic acid carrier is a dendrimer or dendron.

10. The nanoparticle preparation of claim 9, wherein the dendrimer or dendron contains an amine group.

11. The nanoparticle preparation of claim 1, wherein the PEG-lipid is 1,2-dimyristoyl-sn-glycero-3-phosphoethanolamine-N-[methoxy (polyethylene glycol)-2000], 1,2-dimyristoyl-rac-glycero-3-methoxypolyethylene glycol-2000, or any combination thereof.

12. The nanoparticle preparation of claim 1, wherein the PEG-lipid constitutes 1% to 10% by weight of the nanoparticle preparation.

13. The nanoparticle preparation of claim 1, wherein the phospholipid is 1,2-dioleoyl-sn-glycero-3-phosphoethanolamine (DOPE), distearoylphosphatidylcholine (DSPC), or any combination thereof.

14. The nanoparticle preparation of claim 1, wherein the phospholipid constitutes 1% to 25% by weight of the nanoparticle preparation.

15. The nanoparticle preparation of claim 1, wherein the cholesterol or its derivative constitutes 1% to 50% by weight of the nanoparticle preparation.

16. The nanoparticle preparation of claim 1, wherein the metal salt is selected from the group consisting of calcium chloride, magnesium chloride, manganese chloride, barium chloride, copper chloride, zinc chloride, calcium hydroxide, magnesium hydroxide, manganese hydroxide, barium hydroxide, copper hydroxide, zinc hydroxide, calcium sulfate, magnesium sulfate, manganese sulfate, barium sulfate, copper sulfate, zinc sulfate, aluminum chloride, aluminum sulfate, aluminum hydroxide, ferric chloride, ferric sulfate, ferric hydroxide, and any combinations thereof.

17. The nanoparticle preparation of claim 1, wherein the metal salt constitutes 0.01% to 90% by weight of the nanoparticle preparation.

18. A pharmaceutical composition comprising the nanoparticle preparation of claim 1 and a pharmaceutically acceptable carrier.

19. A method for treating or preventing a condition in a subject comprising administering to a subject in need thereof a therapeutically effective amount of the nanoparticle preparation of claim 1.

20. The method of claim 19, wherein the therapeutically effective amount of the nanoparticle preparation or its composition contains the nucleic acid agent in a range from 0.001 ng nucleic acid to 10 mg nucleic acid per kg body weight of the subject.

21. The method of claim 19, wherein the subject is a mammal selected from the group consisting of a human, a non-human primate, a mouse, a rat, a dog, a cat, a horse, and a cow.

22. The method of claim 19, wherein the condition is associated with severe acute respiratory syndrome coronavirus 2 (SARS-COV-2), coronavirus disease 2019 (COVID-19), respiratory syncytial virus (RSV), influenza, Zika virus, influenza virus, tuberculosis, or parasites of the genus Plasmodium.

23. The method of claim 19, wherein the condition is melanoma, non-small cell lung cancer (NSCLC), triple-negative breast cancer (TNBC), type 1 diabetes, rheumatoid arthritis, psoriasis, allergic disease, Danon disease, coronary artery disease (CAD), hypertension, congestive heart failure, myocardial infarction, arrhythmias, cardiac inflammation, or dilated cardiomyopathy.

24. A method of preparing the nanoparticle preparation of claim 1, the method comprising the steps of:
providing an aqueous phase containing the nucleic acid agent,
providing a lipid phase containing the nucleic acid carrier, the PEG-lipid, the phospholipid, and the cholesterol or its derivative, and
mixing the aqueous phase and the lipid phase to form the nanoparticle,
wherein the aqueous or lipid phase contains a metal salt.

25. The method of claim 24, wherein the nucleic acid agent is a polynucleotide, oligonucleotide, a deoxyribonucleic acid (DNA), a complementary DNA (cDNA), a ribonucleic acid (RNA), a replicon RNA (repRNA), a small interfering RNA (siRNA), a microRNA (miRNA), a single strand guide RNA (sgRNA), a messenger RNA (mRNA), or any combination thereof; the nucleic acid carrier is a dendrimer or dendron containing an amine group; the PEG-lipid is 1,2-dimyristoyl-sn-glycero-3-phosphoethanolamine-N-[methoxy (polyethylene glycol)-2000], 1,2-dimyristoyl-rac-glycero-3-methoxypolyethylene glycol-2000, or any combination thereof; and the phospholipid is 1,2-dioleoyl-sn-glycero-3-phosphoethanolamine (DOPE), distearoylphosphatidylcholine (DSPC), or any combination thereof.

26. The method of claim 24, wherein the metal salt is selected from the group consisting of calcium chloride, magnesium chloride, manganese chloride, barium chloride, copper chloride, zinc chloride, calcium hydroxide, magnesium hydroxide, manganese hydroxide, barium hydroxide, copper hydroxide, zinc hydroxide, calcium sulfate, magnesium sulfate, manganese sulfate, barium sulfate, copper sulfate, zinc sulfate, aluminum chloride, aluminum sulfate, aluminum hydroxide, ferric chloride, ferric sulfate, ferric hydroxide, and any combinations thereof.
